(19) United States
(12) Patent Application Publication
(10) Pub. No.: US 2016/0206220 A9
(43) Pub. Date: Jul. 21, 2016

(54) Pulse Photoplethysmogram System for Diabetes Assessment

(71) Applicant: Mark Ries Robinson, Albuquerque, NM (US)

(72) Inventor: Mark Ries Robinson, Albuquerque, NM (US)

(21) Appl. No.: 14/470,927

(22) Filed: Aug. 27, 2014

Related U.S. Application Data
(60) Provisional application No. 61/987,476, filed on May 1, 2014.

(51) Int. Cl. A61B 5/024; A61B 5/00; A61B 5/02

(52) U.S. Cl. CPC A61B 5/02416; A61B 5/02007; A61B 5/7246; A61B 5/0082

(57) ABSTRACT

The present invention provides a method for assessing diabetes in an individual by characterization of a pulse waveform indicative of the response of a portion of the cardiovascular system of the individual to pressure pulses produced during the cardiac cycle, where “assessing diabetes” comprises determining the presence or likelihood of diabetes; the degree of progression of diabetes; a change in the presence, likelihood, or progression of diabetes; a probability of having, not having, developing, or not developing diabetes; the presence, absence, progression, or likelihood of complications from diabetes; or a combination thereof. Embodiments of the present invention comprise determining a measure of arterial compliance of the individual from the pulse waveform; and assessing diabetes based on the measure of arterial compliance.

### BACKGROUND OF INVENTION

Diabetes.

Diabetes mellitus is a major health problem in the United States and throughout the world's developed and developing nations. In 2002, the American Diabetes Association (ADA) estimated that 18.2 million Americans—fully 6.4% of the citizenry—were afflicted with some form of diabetes. Of these, 90-95% suffered from Type 2 diabetes, and 35%, or about 6 million individuals, were undiagnosed. See ADA Report, Diabetes Care, 2003. The World Health Organization (WHO) estimates that 175 million people worldwide suffer from diabetes; Type 2 diabetes also represents 90% of all diagnoses worldwide. Unfortunately, projections indicate that this grim situation will worsen in the next two decades. The WHO forecasts that the total number of diabetics will double before the year 2025. Similarly, the ADA estimates that by 2020, 8.0% of the US population, some 25 million individuals, will have contracted the disease. Assuming rates of detection remain static, this portends that, in less than twenty years, three of every 100 Americans will be ‘silent’ diabetics. It is no surprise that many have characterized the worldwide outbreak of diabetes as epidemic.

Diabetes has a significant impact on individual health and the national economy. U.S. health care costs related to diabetes exceeded $132 billion in 2002. Due to the numerous complications that result from chronic hyperglycemia, these costs were distributed over a wide array of health services. For example, between 5 and 10 percent of all U.S. expenditures in the areas of cardiovascular disease, kidney disease, endocrine and metabolic complications, and ophthalmic disorders were attributable to diabetes. See ADA Report, Diabetes Care, 2003. These economic and health burdens belie the fact that most diabetes-related complications are preventable. The landmark Diabetes Control and Complications Trial (DCCT) established that a strict regimen of glucose monitoring, exercise, proper diet, and insulin therapy significantly reduced the progression of and risk for developing diabetic complications. See DCCT Research Group, N Eng J Med, 1993. Furthermore, the ongoing Diabetes Prevention Program (DPP) has already demonstrated that individuals at risk for diabetes can significantly reduce their chances of contracting the disease by implementing lifestyle changes such a weight loss and increased physical activity. See DPP Research Group, N Eng J Med, 2002. ADA has recommended that health care providers begin screening of individuals with one or more disease risk factors, observing: “If the DPP demonstrates a reduction in the incidence of Type 2 diabetes as a result of one or more of the [tested] interventions, then more widespread screening . . . may be justified”. See ADA Position Statement, Diabetes Care, 2003.

The Fasting Plasma Glucose (FPG) test is one of three accepted clinical standards for the diagnosis of or screening for diabetes. See ADA Committee Report, Diabetes Care, 2003. The FPG test is a carbohydrate metabolism test that measures plasma glucose levels after a 12-14 hour fast. Fasting stimulates the release of the hormone glucagon, which in turn raises plasma glucose levels. In non-diabetic individuals, the body will produce and process insulin to counteract the rise in glucose levels. In diabetic individuals, plasma glucose levels remain elevated. The ADA recommends that the FPG test be administered in the morning because afternoon tests tend to produce lower readings. In most healthy individuals, FPG levels will fall between 70 and 100 mg/dl. Medications, exercise, and recent illnesses can impact the results of this test, so an appropriate medical history should be taken before it is performed. FPG levels of 126 mg/dl or higher indicate a need for a subsequent retest. If the same levels are reached during the retest, a diagnosis of diabetes mellitus is typically rendered. Results that measure only slightly above the normal range may require further testing, including the Oral Glucose Tolerance Test (OGTT) or a postprandial plasma glucose test, to confirm a diabetes diagnosis. Other conditions which can cause an elevated result include pancreatitis, Cushing's syndrome, liver or kidney disease, eclampsia, and other acute illnesses such as sepsis or myocardial infarction.

The OGTT is the clinical gold standard for diagnosis of diabetes despite various drawbacks. After presenting in a fasting state, the patient is administered an oral dose of glucose solution (75 to 100 grams of dextrose) which typically causes blood glucose levels to rise in the first hour and return to baseline within three hours as the body produces insulin to normalize glucose levels. Blood glucose levels may be measured four to five times over a 3-hour OGTT administration. On average, levels typically peak at 160-180 mg/dl from 30 minutes to 1 hour after administration of the oral glucose dose, and then return to fasting levels of 140 mg/dl or less within two to three hours. Factors such as age, weight, and race can influence results, as can recent illnesses and certain medications. For example, older individuals will have an upper limit increase of 1 mg/dl in glucose tolerance for every year over age 50. Current ADA guidelines dictate a diagnosis of diabetes if the two-hour post-load blood glucose value is greater than 200 mg/dl on two separate OGTTs administered on different days.

Glycated Hemoglobin (hemoglobin A1c, A1c or HBA1c) is also recommended by the ADA for the diagnosis of or screening for diabetes effective 2010. HBA1c is a form of hemoglobin that influenced by the average plasma glucose concentration over life of the red blood cell. It is formed in a non-enzymatic glycation pathway by hemoglobin's exposure to plasma glucose. Normal levels of glucose produce a normal amount of glycated hemoglobin. As the average amount of plasma glucose increases, the fraction of glycated hemoglobin increases in a predictable way. This serves as a marker for average blood glucose levels over the previous months prior to the measurement, and therefore serves as a marker for diabetes. An HbA1c level greater than or equal to 6.6% is diagnostic of diabetes.

In addition to these diagnostic criteria, the ADA also recognizes two ‘pre-diabetic’ conditions reflecting deviations from euglycemia that, while abnormal, are considered insufficient to merit a diagnosis of diabetes mellitus. An individual is said to have ‘pre-diabetes’ when a single FPG test falls between 100 and 126 mg/dl or Hba1c is between 5.7 to 6.4%, or when the OGTT yields 2-hour post-load glucose values between 140 and 200 mg/dl, a diagnosis of ‘pre-diabetes’ is typically rendered. Both of these conditions are considered risk factors for diabetes.

The need for pre-test fasting, invasive blood draws, and repeat testing on multiple days combine to make the OGTT, A1c and FPG tests inconvenient for the patient and expensive to administer. In addition, the diagnostic accuracy of these tests leaves significant room for improvement. See, e.g., M. P. Stern, et al., Ann Intern Med, 2002, and J. S. Yudkin et al., BMJ, 1990. Various attempts have been made in the past to avoid the disadvantages of the FPG and OGTT in diabetes screening. For example, risk assessments based on patient history and paper-and-pencil tests have been attempted, but such techniques have typically resulted in lackluster diagnostic accuracy.

A reliable, convenient, and cost-effective means to screen for diabetes mellitus is needed. Also, a reliable, convenient, and cost-effective means for measuring effects of diabetes could help in treating the disease and avoiding complications from the disease.

Arterial Compliance.

The classic definition by Spencer and Denison of compliance (C) is the change in arterial blood volume (ΔV) due to a given change in arterial blood pressure (ΔP). So, C=ΔV/ΔP.

Arterial compliance can be an index of the elasticity of large arteries such as the thoracic aorta. Arterial compliance is an important cardiovascular risk factor. Compliance generally diminishes with age. Age affects the wall properties of central elastic arteries (aorta, carotid, iliac) in a different manner than in muscular arteries (brachial, radial, femoral, popliteal). With increasing age the pulsatile strain breaks the elastic fibers, which are replaced by collagen. On the other hand, there is only little alteration of distensibility of the muscular, i.e. distal, arteries with age.

In healthy and compliant arteries the pressure waves (generated by the left ventricle) travel through the arterial tree and are reflected at multiple peripheral sites. As a result, the arterial pressure waveform at any site is a combination of the forward travelling waveform and the backward (or reflection) waveform. The two waveforms merge during diastole and augment coronary perfusion. With aging, the arterial wall thickens and the arteries get stiffer. As a result, the pressure waves travel faster and the reflected pressure wave returns during the systolic phase, increasing systolic pressure and thus increasing left ventricular load.

Endothelial dysfunction results in reduced compliance (increased arterial stiffness), especially in the smaller arteries. This is characteristic of patients with hypertension. However, it may be seen in normotensive patients (with normal blood pressure) before the appearance of clinical hypertension. Reduced arterial compliance is also seen in patients with diabetes and also in smokers. It is actually a part of a vicious cycle that further elevates blood pressure, aggravates atherosclerosis (hardening of the arteries), and leads to increased cardiovascular risk. Arterial compliance can be measured by several techniques. Most of them are invasive and are not clinically appropriate.

Conventional Measurement of Arterial Compliance.

The most common method for determining arterial compliance is the determination of pulse wave velocity. In cardiovascular research and clinical practice, Pulse wave Velocity (PWV) refers to the velocity of pressure pulses that propagate along the arterial tree. In particular, those pressure pulses generated during left ventricular ejection: at the opening of the aortic valve, the sudden rise of aortic pressure is absorbed by the elastic aorta walls. Subsequently a pulse wave naturally propagates along the aorta exchanging energy between the aortic wall and the aortic blood flow (FIG. 2).

Modifications of the arterial wall compliance or stiffness will induce changes at the velocity at which pressure pulses travel along it. The determination of aortic PWV is a direct measurement of aortic stiffness and is considered to be the gold standard of arterial stiffness measurements. Aortic PWV is a measure of the speed of the arterial pressure waves travelling along the aortic and aorto-iliac pathway. Higher arterial pressure wave velocity is indicative of stiffer arteries. See FIG. 3. FIG. 3 is an illustration of aortic PWV, which is defined as the average velocity of a pressure pulse when travelling from the aortic valve, through the aortic arc until it reaches the iliac bifurcation.

In general, given an arterial segment of length D, we define its PWV as:

$$P W V = \frac{D}{P T T}$$

where PTT is the so-called Pulse Transit Time, i.e. the time that a pressure pulse will require to travel through the whole segment. Formally PTT is defined as:

PTT=PAT_{d}=PATₚ

where PATₚ corresponds to the arrival time of the pressure pulse at the proximal (closer to the heart) extremity of the artery, and PAT_{d} corresponds to the arrival time of the pressure pulse at its distal (distant to the heart) extremity.

Stiffness=Distance/(PTT)

The most common technique for measuring PWV is through the non-invasive method of applanation tonometry. In general, measurements are performed by recording pressure waveforms at the carotid artery followed by the femoral artery, with an ECG signal being recorded simultaneously. PWV is calculated using the mean time difference and the arterial path length between the two recording sites.

### SUMMARY OF THE INVENTION

Embodiments of the present invention provide a reliable, convenient, and cost-effective means to screen for diabetes mellitus is needed. The present invention provides a method for assessing diabetes in an individual by characterization of a pulse waveform indicative of the response of a portion of the cardiovascular system of the individual to pressure pulses produced during the cardiac cycle, where “assessing diabetes” comprises determining the presence or likelihood of diabetes; the degree of progression of diabetes; a change in the presence, likelihood, or progression of diabetes; a probability of having, not having, developing, or not developing diabetes; the presence, absence, progression, or likelihood of complications from diabetes; or a combination thereof. Embodiments of the present invention comprise determining a measure of arterial compliance of the individual from the pulse waveform; and assessing diabetes based on the measure of arterial compliance.

### BRIEF DESCRIPTION OF THE DRAWINGS

FIG. 1 is an illustration of the natural history of Type 2 diabetes.

FIG. 2 is an illustration of the genesis of pressure pulses.

FIG. 3 is an illustration of aortic Pulse Wave velocity measurement.

FIG. 4 is an illustration of the results of analysis of three different measures of compliance in three groups with different glucose control.

FIG. 5 is an illustration of relative changes in peripheral arterial stiffness and central arterial stiffness as a function of glucose control

FIG. 6 is an illustration of pulse waves.

FIG. 7 shows a configuration of peripheral compliance assessment.

FIG. 8 shows regression curves showing the effect on age on Pulse Wave Velocity.

FIG. 9 shows a typical method for the calculation of augmentation index.

FIG. 10 shows the change in contour and amplitude of pressure waves recorded in the radial artery in normal subjects between the first and eight decades of life.

FIG. 11 shows an example of the pulses used for analysis.

FIG. 12 is an illustration of receiver-operator characteristic.

FIG. 13 is an illustration of receiver-operator characteristic relating to embodiments of the present invention.

FIG. 14 illustrates that as the absorption of the light increases, detected light tends to come from the shorter paths; using the results of a random walk model for photons scattering through tissue media.

FIG. 15 provides the estimated path length distributions for the three scenarios shown in FIG. 14.

FIG. 16 is a schematic depiction of a system that uses an optical sampling method based upon cross polarization to preferentially select those photons that have been scattered by the tissue.

FIG. 17 shows examples of the types of measurement artifacts and the extremes in physiological variation that can be seen in PPG recordings.

FIG. 18 shows an example embodiment with a multi-wavelength source and integrating device.

FIG. 19 shows the absorbance characteristics of blood.

FIG. 20 shows a pictorial representation of DOP over the wavelength range of 580 to 700 nm.

FIG. 21 shows a pictorial representation of DOP over the wavelength range of 580 to 700 nm.

FIG. 22 is an illustration of the result from expanding the concept to all wavelengths.

FIG. 23 is an illustration of numerical aperture.

FIG. 24 shows a reconfiguration of the system of FIG. 18 but where the polarization aspects of the system have been replaced by methods for numerical aperture control.

FIG. 25 is a schematic illustration of an example embodiment.

FIG. 26 is a schematic illustration of an example embodiment.

FIG. 27 is an illustration of a portion of FIG. 11.

FIG. 28 is a schematic illustration of an example embodiment.

FIG. 29 is a schematic illustration of an example embodiment.

FIG. 30 shows the relationship between relative volume and transmural pressure.

FIG. 31 is an illustration of aortic pulse wave velocity and volume removed.

### DESCRIPTION OF THE INVENTION

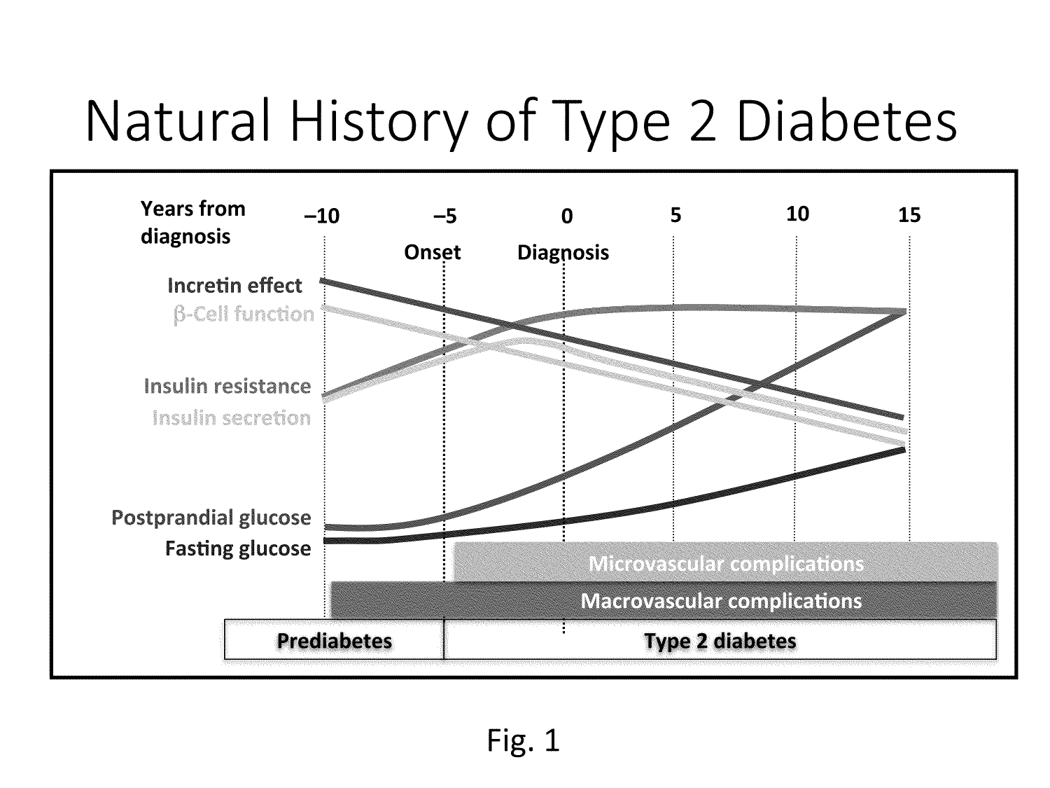

The above diabetes assessment testing methods are based upon the body's inability to control glucose either during a fasting state or after being subjected to a glucose load. However, the true pathophysiology of diabetes contains a number of other physiological markers that are predictive of prediabetes and diabetes. The initiation of diabetes begins with an increase in insulin resistance and impairments in b-cell function. Over time relative insulin deficiency occurs as well as excessive glucagon production leading to overproduction of endogenous glucose in the liver. These malfunctions in glucose control eventually lead to postprandial hyperglycemia and then elevations in fasting blood glucose levels. These relationships are shown graphically in FIG. 1, obtained from the American Association of Clinical Endocrinologists Diabetes Research Center, Adapted from Holman R R. Diabetes Res Clin Pract. 1998; 40(suppl):S21-S25; Ramlo-Halsted B A, Edelman S V. Prim Care. 1999; 26:771-789; Nathan D M. N Engl J Med. 2002; 347:1342-1349; UKPDS Group. Diabetes. 1995; 44:1249-1258.

In addition to these changes in the ability to regulate glucose, additional changes occur with respect to the vascular system. Examination of FIG. 1 shows that macrovascular changes as well as microvascular changes occur prior to typical diagnosis. It is especially important to note that macrovascular changes occur very early in the natural progression of Type II diabetes. As it relates to diabetes assessment, the identification of these macrovascular changes can create a diabetes assessment test that can identify the disease earlier and would result in a better sensitivity.

The present invention provides an ability to detect vascular changes associated with pre diabetes and diabetes such that an improved diabetes test is possible. Specifically, the invention relates to the determination of arterial compliance as a method for diabetes assessment.

The invention relates to a new method for diabetes screening that is based not upon glucose measurements but rather upon physiological characteristics of the cardiovascular system. The physiological parameter being used for assessment of diabetes status is arterial stiffness. Arterial stiffness can be evaluated by analysis of an individual's pulse waveform. The proposed system determines pulse wave velocity characteristics by evaluating pulse photoplethysmography (PPG) data through evaluation methods that provide information on aortic stiffness. The system utilizes a novel data acquisition system based upon polarization techniques that enable measurement of photons that have traveled different paths, with preferential selection of those photons that have been highly scattered. The resulting data has improved sensitivity for pulse variations and can be processed in a manner that mitigates motion artifacts. The differentiated processing method is based upon the Degree of Polarization. The resulting multi-wavelength Degree of Polarization measurement can be evaluated over time to create an improved signal-to-noise and motion-compensated PPG signal. The resulting diabetes assessment is done through a new processing method that enables assessment of both central artery compliance and peripheral artery compliance. The assessment of diabetes from the PPG signal is conducted by methods that depart from the prior art by using methods that effectively incorporate all information available in the pulse waveform including time features, amplitude features and shape features; and enable compensation for parameters that are known to influence arterial stiffness, such as chronological age.

The resulting method requires no patient compliance such as fasting, does not require a blood draw, provides immediate results and is based upon physiological parameters that are leading indicators for the development or presence of diabetes.

As the invention incorporates multiple inventive steps, these will be explained in detail. Additionally, it is recognized that each improvement can be used independently or in conjunction with other improvements to create a diabetes assessment system that is a dramatic improvement over conventional approaches in terms of cost, convenience and performance.

As used herein, “diabetes assessment” includes determining the presence or likelihood of diabetes; the degree of progression of diabetes; a change in the presence, likelihood, or progression of diabetes; a probability of having, not having, developing, or not developing diabetes; the presence, absence, progression, or likelihood of complications from diabetes. “Diabetes” includes a number of blood glucose regulation conditions, including Type I, Type II, and gestational diabetes, other types of diabetes as recognized by the American Diabetes Association (See ADA Committee Report, Diabetes Care, 2003), hyperglycemia, impaired fasting glucose, impaired glucose tolerance, and pre-diabetes. As used herein, photoplethysmography (PPG) is an optical measurement technique that can be used to detect blood volume changes in tissue or has a signal that is related to the cardiac cycle. Arterial compliance refers to the general ability of a blood vessel wall to expand and contract passively with changes in pressure and includes a multitude of metrics and terms used to refer to related properties such a stiffness, elastance, Young's modulus, elastic modulus and other parameters that define the general relationship between increasing volume with increasing transmural pressure or the tendency of a hollow organ to resist recoil toward its original dimensions on application of a distending or compressing force.

Diabetes Changes Arterial Compliance.

Diabetes mellitus is one of the major cardiovascular risk factors and has been associated with premature atherosclerosis. There are numerous studies showing that both patients suffering from Type 1 diabetes and Type 2 diabetes have an increased arterial stiffness compared to controls. The increase in arterial stiffening in patients with Type 1 and Type 2 diabetes mellitus is evident even before clinical micro- and macrovascular complications occur, being already present at the stage of impaired glucose tolerance.

Schram, Miranda T., et al. “Increased central artery stiffness in impaired glucose metabolism and Type 2 diabetes the Hoorn Study.” Hypertension 43.2 (2004): 176-181, and Stehouwer, C. D. A., R. M. A. Henry, and I. Ferreira. “Arterial stiffness in diabetes and the metabolic syndrome: a pathway to cardiovascular disease.” Diabetologia 51.4 (2008): 527-539, have studied and published on the relationship between arterial compliance and the development of diabetes. A large body of evidence supports the concept of increased arterial stiffness in Type 1 diabetes. This is an early phenomenon that occurs before the onset of clinically overt micro- or macrovascular disease and arterial stiffness is further enhanced in the presence of microvascular complications (e.g. nephropathy, microalbuminuria or retinopathy). Similar findings have been reported with regard to pulse pressure: in individuals with Type 1 diabetes an increase in pulse pressure can be detected as early as the third and fourth decade of life, i.e. there is accelerated arterial ageing, and the age-pulse pressure relationship is even steeper in the presence of microvascular complications. In aggregate, the data support the concept of accelerated arterial ageing in Type 1 diabetes.

Similar to Type 1 diabetes, a large body of evidence supports the concept of increased arterial stiffness in Type 2 diabetes. Increased arterial aging is an early phenomenon associated with impaired glucose metabolism state (i.e. impaired fasting glucose and/or impaired glucose tolerance or more currently referred to as pre-diabetes).

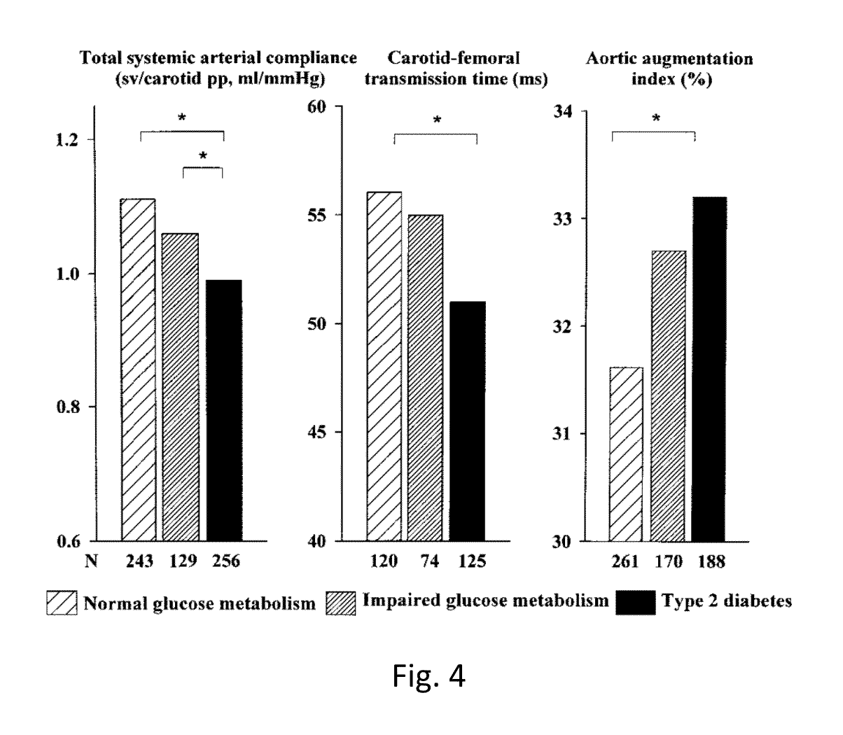

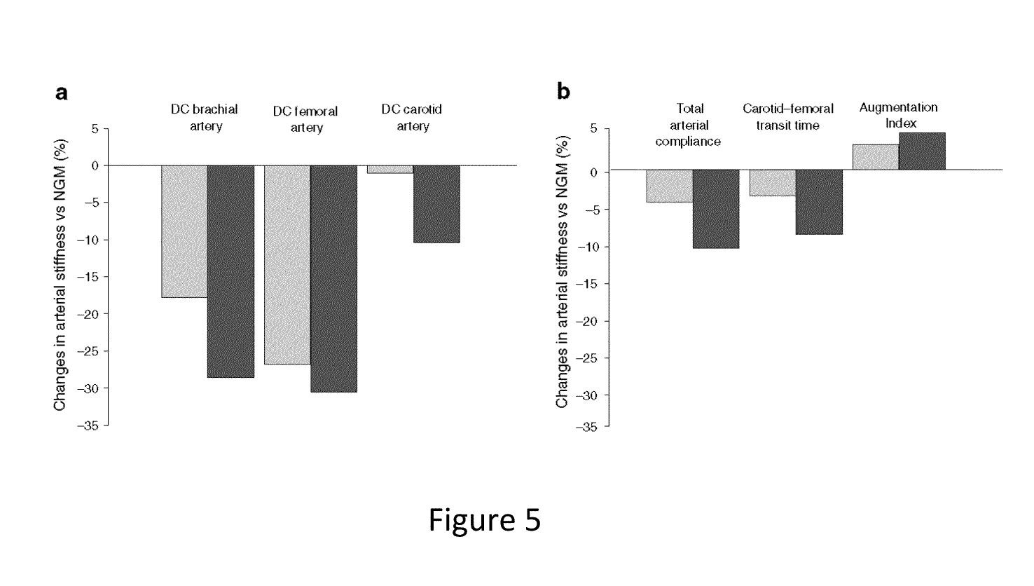

The work of Schram et al. examined the three different measures of compliance in three groups with degrees of glucose control; normal, impaired glucose metabolism and Type 2 diabetes. The results of this analysis are shown in FIG. 4. Examination of FIG. 4 shows a clear relationship between increasing diabetes severity and decreased arterial compliance. FIG. 5 shows the information via an analysis to show where arterial stiffness changes occur. FIG. 5 shows relative changes in peripheral arterial stiffness (local arterial distensibility coefficients [DC] of the brachial, femoral and carotid arteries) (a), and central arterial stiffness (systemic compliance, carotid-femoral transit time and aortic augmentation index) (b) in individuals with Type 2 diabetes and impaired glucose metabolism.

FIG. 5 demonstrates arterial stiffness changes in both the central arteries as well as the peripheral arteries, however there exists uncertainty regarding the possible preferential stiffening of peripheral over central arteries. The currently available literature does not clearly define a preferential position.

Despite this uncertainty in the preferential stiffening, the following summary points are clearly supported. Arterial stiffness is increased in Type 1 diabetes and is an early phenomenon that occurs before the onset of clinically overt micro- or macrovascular complications. Arterial stiffness is increased in Type 2 diabetes and is an early phenomenon that occurs in the impaired glucose metabolism state. The presence of micro- and macrovascular complications is associated with a further increase in arterial stiffness. Arterial stiffness is also increased in the metabolic syndrome and in insulin-resistant states and subtle changes in metabolic variables (not fully developed diabetes) affect arterial stiffness from an early age. Diabetes is a disease of accelerated arterial aging, as shown by stiffer arteries and consequent steeper increases in pulse pressure with age in these subjects.

Reflected Waves in Peripheral Arteries.

As discussed above, the arterial pulse is a combination of the pulse generated by the heart as well as reflected waves. Wave reflection is a consequence of the cardiovascular design and termination of highly conductive arteries in highly resistive arterials. The same phenomenon that causes mean pressure to fall abruptly over a short length in the arterioles is also responsible for reflection of the pressure wave that approaches the arterioles. In technical terms, an impedance mismatch causes wave reflections. There are a myriad of reflection sites within the body at different distances from the heart, since they correspond to arterial terminations in different tissues. The reflected waves observed in a peripheral pressure pulse are the result of multiple reflections from within the systemic circulation. While arterioles are the major source of wave reflection in humans, reflection can arise at any point of impedance mismatch.

The analysis of the arterial pulse waveform has been the subject of many studies and a significant body of work that has been published over the past 40 or so years. The composition of the arterial pulse wave has investigated through the use of invasive central artery manometers to track mechanical events such as heart contractions and pressure pulse reflections in the central arterial tree, to the arterial periphery. These studies (Latham R D, Westerhof N, Sipkema P, Rubal B J, Reuderink P, Murgo J P: Regional wave travel and reflections along the human aorta: a study with six simultaneous micromanometric pressures. Circulation 1985, 72:1257-69, Ting C T, Chang M S, Wang S P, Chiang B N, Yin F C: Regional pulse wave velocities in hypertensive and normotensive humans. Cardiovasc Res 1990, 24(11):865-72, and KHz J, Seba P: Force plate monitoring of human hemodynamics) have confirmed the existence of two major reflection sites in the central arteries. The first reflection site is the juncture between thoracic and abdominal aorta, which is marked by a significant decrease in diameter and a change in elasticity and the second site arises from the juncture between abdominal aorta and common iliac arteries. These reflection sites will be respectively referred to as the “renal” and the “iliac” reflection sites.

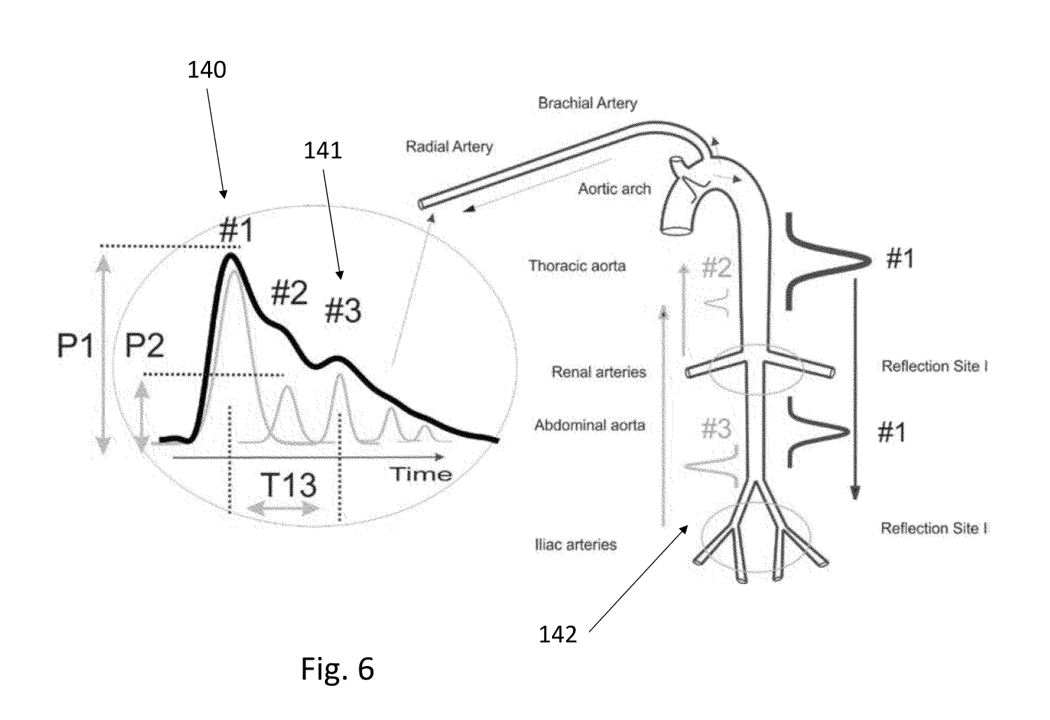

A consequence of these impedance mismatch sites is reflected arterial pressure pulses that counter-propagate to the direction of the single arterial pressure pulse. Referring to FIG. 6, the “downward” travelling primary pressure pulse #1 gives rise to the “upward” travelling #2 and #3 pulses that are respectively due to the renal and the iliac reflection sites on which the #1 pulse impinged.

As these reflected #2 and #3 pressure pulses reach the aortic arch, they will enter the subclavian arteries and head into the arterial periphery of the arm, following the #1 pressure pulse that has entered the arm arteries. The #2 and #3 pulses will also enter the arm arteries following a time delay due to the time associated with transversely the aorta.

The #2 pulse is commonly known as the “second systolic” peak and is due to the renal impedance mismatch and typically enters the arm with a delay of between 70-140 milliseconds. The pulse labeled #3 in FIG. 6 is the much larger “iliac reflection”, which follows the #1 pulse at delays of 180 to 400 milliseconds.

The primary wave and the two significant sources of distinct impedance mismatch served to create three component pulses in the pressure pulse envelope that is observed in the arterial periphery of the arm, such as at the radial or digital arteries. There are additional reflected waves present from multiple arteriole termination and other re-reflections but this simplified model can be used to explain the structure of the digital pressure pulse. Simplistically, the brachial/radial arterial pulse can be reasonably explained by the interaction of the primary left ventricular ejection pressure pulse with two aortic reflection sites and an almost constant background noise of arteriole reflections.

New Method for Determination of Aortic Stiffness by Pulse Wave Analysis.

With an understanding of diabetes and arterial compliance as well as reflected wave propagation in the body, a new method for determination of Aortic Transit Time and effectively aortic stiffness can be defined. The aorta is significantly different than the peripheral arteries due to the fact that the aorta does not have a significant smooth muscular layer. Therefore, issues associated with vasoconstriction or vasodilation have less influence on blood flow characteristics in the aorta then in peripheral arteries. Therefore, the characteristics of aortic compliance, resistance, etc. are primarily determined by the biomechanical properties of the arterial wall. These biomechanical properties determine the overall arterial compliance in the propagation speed of a pressure pulse traveling on an artery and is inversely related to the arterial compliance, i.e. the more compliant the artery the slower the pulse will propagate. The so-called Moens-Korteweg equation:

$$c = P W V = \sqrt{\frac{{hE}_{inc}}{2 ρ R}}$$

Where, where h is the arterial wall thickness, E_{inc} is the incremental elastic modulus, r is the lumen radius, and ρ is the blood density.

Accordingly, the speed of propagation of a pressure pulse along the arterial wall depends on:
the biomechanics properties of the wall: and in particular its stiffness E_{inc} or Young's modulus,
the geometry of the wall, and in particular its thickness h and radius R,
and the density of blood.

Even though the derivation of the Moens-Korteweg model relies on several (and severe) simplifications, it provides an intuitive insight on the propagation phenomenon in arteries predicting that the stiffer the artery (increased E_{inc}) the faster a pressure pulse will propagate along it. Therefore, for large elastic arteries such the aorta where the thickness to radius ratio is almost invariable, PWV is expected to carry relevant information related to arterial stiffness.

With an awareness of the histopathology associated with the aorta as well as the relationship between pulse wave propagation and arterial stiffness, it is desirable to obtain a stiffness metric that is specific for the aorta.

Consider FIG. 6 where a pulse measurement system is positioned at the end of the radial artery, such as at the wrist or on a digit. The primary wave will be influenced by systolic ejection as well as propagation down the brachial/radial artery. In contrast, the renal reflected wave as well as the iliac reflected wave will contain information regarding transport through the aorta as well as information associated with having traveled the brachial/radial artery. Therefore, the information content associated with these three waveform peaks can be used to access arterial compliance and specifically aortic compliance.

The time difference between arrival of the primary wave and the reflected waves is proportional to aortic pulse wave velocity when normalized for the distance traveled. Therefore it is possible to determine the time needed for a wave to travel down and up the aorta, referred to as the Aortic Travel Time (ATT). The following equations show the specific calculation needed for the determination of ATT as well as the determination of aortic stiffness.

Pulse wave velocity is defined as

$$P W V = \frac{D}{P T T}$$

Where

D is the length of arterial segment
PTT is the pulse transit time to travel Length D

Consider the Primary Pulse Wave (see FIG. 6, 140)

$$P W {V}_{( I )} = \frac{Arm Length}{P A {T}_{H} - P A {T}_{F ( 1 )}}$$

Where

PAT_{H}=Pulse Arrival Time at the Heart (time wave exits the heart)

### PAT_{F(1)}=Pulse Arrival Time at the Finger of First Wave

For the Iliac Reflected Wave (141 in FIG. 6)

$$P W {V}_{( 3 )} = \frac{{D}_{Aorta} + {D}_{Aorta} + {D}_{Arm}}{P A {T}_{H} + P A {T}_{F ( 3 )}}$$

D_{Aorta}=Distance of the heart to Iliac bifurcation (FIG. 6, 142)

PAT_{F(3)}=Pulse Arrival Time at Finger of 3^{rd} Wave (FIG. 6, 141)

For the determination of Aortic stiffness, the equations need to be solved for PWV_{(A)}, Pulse Wave Velocity in the Aorta.

A compliance metric can then be calculated by:

$$Aortic Compliance ∝ \frac{1}{P W {V}_{( A )}}$$

For Reflected Waves (3)

Total Time=T_{Down Aorta}+T_{Up Aorta}+T_{Down Arm}

Therefore,

$$P A {T}_{H} - P A {T}_{F ( 3 )} = ( {T}_{Down} + {T}_{Up} ) + P A {T}_{H} + P A {T}_{F ( 1 )}$$

$${T}_{Down} + {T}_{Up} = ( P A {T}_{H} - P A {T}_{F ( 3 )} ) - ( P A {T}_{H} - P A {T}_{F ( 1 )} )$$

$$Aorta Transit Time = P A {T}_{F ( 1 )} - P A {T}_{F ( 3 )}$$

$$Aorta Transit Time = \frac{( Aorta Length \times 2 )}{P W {V}_{Aorta}}$$

$$P W {V}_{( Aorta )} = \frac{( Aorta Length \times 2 )}{P A {T}_{F ( 1 )} - P A {T}_{F ( 3 )}}$$

Summary

$$P W {V}_{Aorta} ∝ Aortic Compliance$$

$$P W {V}_{Aorta} ∝ \frac{1}{Aortic Stiffness}$$

The above equations show that a new measure specific for aortic compliance can be calculated based upon the primary pulse and the use of the reflected pulses. The process enables the determination of Aortic Transit Time. The distance of travel can be measured physically, estimated by total height, of measured from known landmarks such as a xiyphoid process to the navel. The xiphoid process is at the lower portion of the sternum, or breastbone and can be easily palpated.

The above methods reference the use of the third peak in the pulse waveform but other peaks can be used, including the second peak commonly associated with the renal artery reflection.

Mechanism for Determination of Peripheral Stiffness by Pulse Wave Analysis.

For a true determination of a given individual's cardiovascular condition and diabetes state, it can be desirable to derive a measurement more specific for peripheral compliance. Based upon the current literature, the pathophysiology of changes due to diabetes in elastic arteries a.k.a. central arteries versus peripheral or muscular arteries can be different.

The process of obtaining a measurement specific for peripheral compliance can be obtained by obtaining two pressure waveforms concurrently at slightly different distances from the heart. As examples, a wrist to finger measurement or an index to pinky finger relationship can provide the necessary information. Although the overall distance in measurement sites is significantly smaller than that used historically for pulse wave velocity determinations, increased sampling frequency and the ability to effectively synchronize measurement methodologies make such a measurement possible. The determination of a peripheral pulse wave velocity measurement can be used in conjunction or independently with a central pulse wave velocity for diabetes assessment. The combined information may provide insight in cardiac risk, hypertension, disease progression, and effectiveness of treatment.

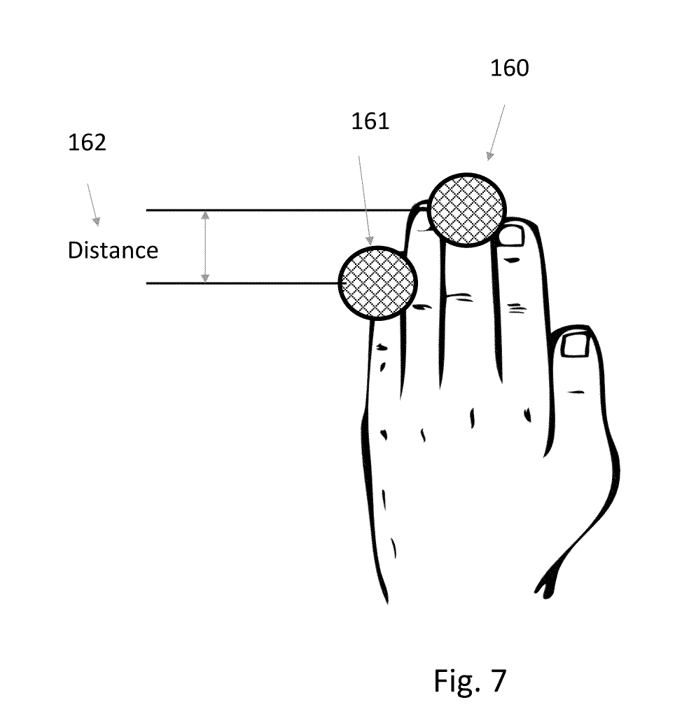

FIG. 7 shows a configuration of peripheral compliance assessment. Site 160 is a PPG measurement sight that is more distal than the site 161, creating distance difference 162. Standard PWV determinations can be applied on the resulting data.

Improved Performance by Use of Ancillary Information.

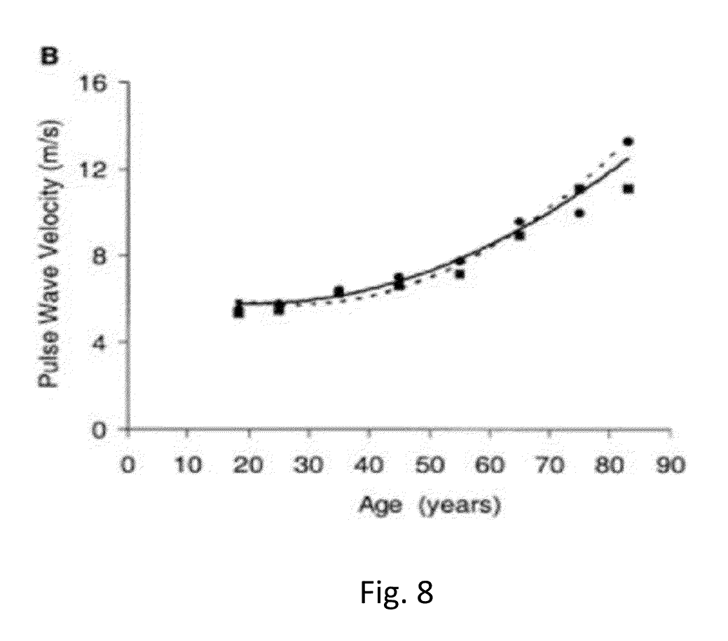

Arterial compliance as described above is clearly influenced by the development of diabetes and there is a strong relationship between aortic compliance and deteriorating glucose metabolism status. It is recognized, that additional factors influence arterial compliance. A well-recognized change in arterial compliance occurs with age. FIG. 8 shows regression curves showing the effect on age on Pulse Wave Velocity for males (circles, solid lines) and females (squares, dashed lines), from: McEniery C M, Yasmin, Hall I R, et al. Normal vascular aging: differential effects on wave reflection and aortic pulse wave velocity: the Anglo-Cardiff Collaborative Trial (ACCT). J Am Coll Cardiol 2005, 46:1753-1760.) Therefore, performance improvements can be obtained by effectively normalizing out or compensating for known influences of arterial compliance. In very simple terms, the diagnostic process can utilize an age as an input variable and effectively compensate for the normal aging process.

With an objective of creating a high performance screening device, additional ancillary information can be utilized by the algorithm to normalize, compensate, or adjust for these known influences. Example candidate ancillary parameters include but are not limited to age, hypertension, duration of hypertension, height, weight, waist size, size, heart rate, need arterial pressure, systolic pressure, diastolic pressure, creatinine, smoking, hypertensive medications, cholesterol, low-density lipoproteins, high density lipoproteins, albumin, plasma homocysteine, smoking duration, triglycerides, alcohol consumption, ethnicity, C-reactive protein, gender, hemoglobin, hematocrit and urea.

In use, these pieces of ancillary information can be entered at the time of use, pulled from an electronic medical record, or in some other manner introduced into the algorithm for appropriate compensation. Additionally, these variables may be used to adjust a numerical output.

Pulse Wave Analysis.

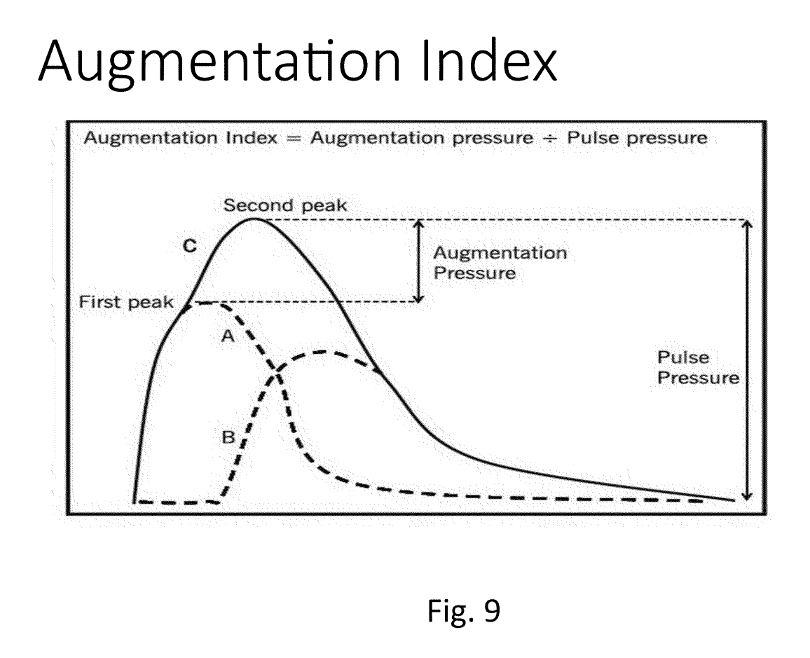

It is important to recognize that pulse wave velocity is the most common metric for determining arterial stiffness. However, there exist several other approaches that quantify other elements of the arterial pulse wave and are broadly classified here as pulse wave analysis. Analysis of the aortic pressure waveform provides a measure of central blood pressure and indices of systemic arterial stiffness, such as Augmentation Pressure (AP) and Augmentation Index (Alx). These parameters are rather simplistic methods that use peak heights or ratios of peak heights for the determination of various parameters. FIG. 9 shows a typical method for the calculation of augmentation index. In the figure: central aortic waveform and augmentation index (Alx); (A) forward wave; (B) reflected waveform; (c) summation waveform as the result of early wave reflection in a patient with stiff arteries.

An important aspect of the invention is the concurrent use of time separation information in the form of pulse wave velocity and also to effectively utilize all information in the pulse wave, including amplitude and shape information.

Demonstration of Improved Pulse Wave Analysis.

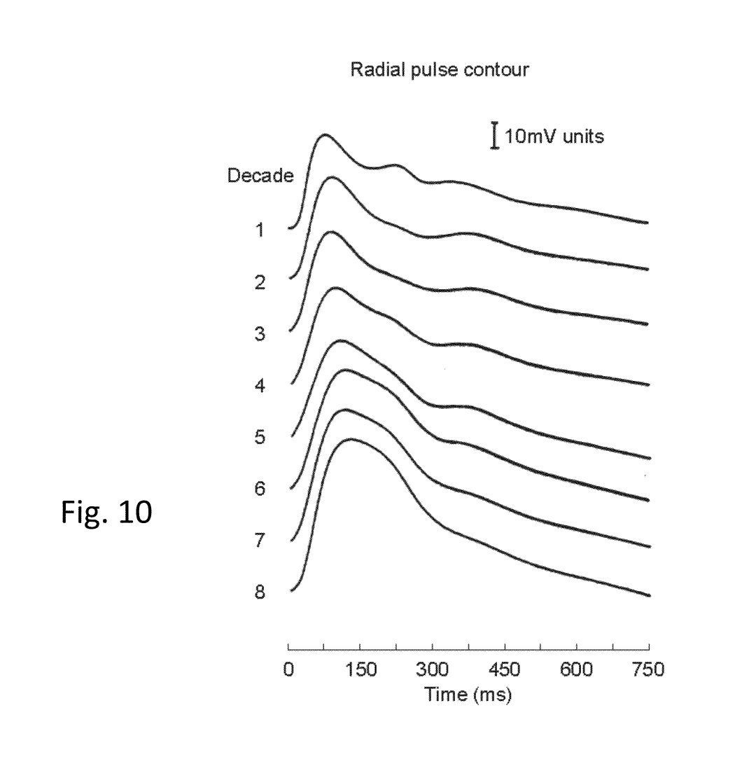

Determination of Aortic Travel Time and the associated aortic pulse wave velocity provides information regarding aortic compliance. The determination of this information can be solely based upon time differences between peak heights. In addition to pulse arrival time differences, a significant amount of additional information regarding aortic compliance is available through analysis of the amplitude of the wave, the frequency components of the wave, and the overall shape of the wave. Examination of FIG. 10 shows some of the difficulty associated with a system based upon only peak arrival determinations. FIG. 10 shows the change in contour and amplitude of pressure waves recorded in the radial artery in normal subjects between the first and eight decades of life. Examination of the pulse waveform associated with the eighth decade of life shows that the reflected waves are so overlapped with the primary wave that an independent determination of peak heights would be difficult.

As stated previously, prediabetes and diabetes lead to accelerated aging of the vascular system. Therefore, shape-based methods based using distance metric calculations or other methodologies can be used to determine the “effective age” of a recorded pulse profile. For example, if a 40-year-old individual were to generate a pulse waveform more consistent with that of a 60-year-old individual it can be indicative of significant arterial aging and compliance changes.

In “Non-invasive estimate of blood glucose and blood pressure from a photoplethysmograph by means of machine learning techniques”, Monte-Moreno, Enric, Artificial Intelligence in Medicine, Volume 53, Issue 2, 127-138, authors use a number of techniques to clean, filter, and extract features from photoplethysmographs. The approach filters the signal from a train of photoplethysmographs relating to the signal generated by a train of heatbeat pulses a variety of metrics. Energy, Qi-Zeng Energy, Entropy crossing rate for each signal are computed by a FFT transform. All computed quantities are collected into a vector of features, one per each case, that are used to train several classification approaches (Linear mode, Neural Networks, Support Vector Machines, Classification and Regression Trees and Random Forest) to determine blood pressure or blood glucose. The method is not used for determination of pulse wave velocity, arterial compliance, or any assessment of diabetes state.

In “Multi-Gaussian fitting for pulse waveform using Weighted Least Squares and multi-criteria decision making method”, Wang, Lu et al., Computers in Biology and Medicine, Volume 43, Issue 11, 1661-1672, authors use well known techniques of fitting a number of Gaussian curves to represent photoplethysmograph signals. The approach decomposes the physiological signal generated from an appropriate instrument into a number of Gaussian curves. The sum of Gaussian curves is fitted to the physiological curve by mean of Weighted Least Squares. Goodness-of-fit is estimated and studied in the paper. The paper provides a mechanism for fitting does not pulse waveforms but does not articulate a use for the fitted parameters. The approach is limited by assuming that the signal is composed of only Gaussian curves. Thus, no relationship is defined between these Gaussian curve fits and the desired measurement parameter of arterial stiffness.

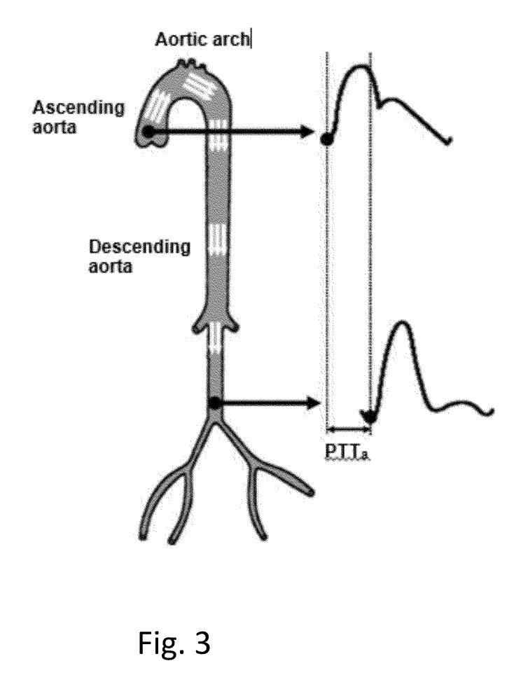

In “Arterial stiffness estimation based photoplethysmographic pulse wave analysis”, Matti Huotari et al., Proc. SPIE 7376, Laser Applications in Life Sciences, 73760L (Nov. 24, 2010), authors used signal generated from photoplethysmograph devices, and analyzed them by decomposing the signal into a small number (five) of component functions fitted by nonlinear least square minimization with the Levenberg-Marquart approach. The fitting errors shown in the publication, FIGS. 3, 4 and 5 show significant residual error, especially during the systolic phase. Despite the publication's title, no true relationship is shown between measured parameters and arterial stiffness. The paper correctly assumes some relationship with age and arterial stiffness and does show general trends associated age and the calculated parameters. Again, no direct measure of arterial stiffness is presented and no association with diabetes is articulated.

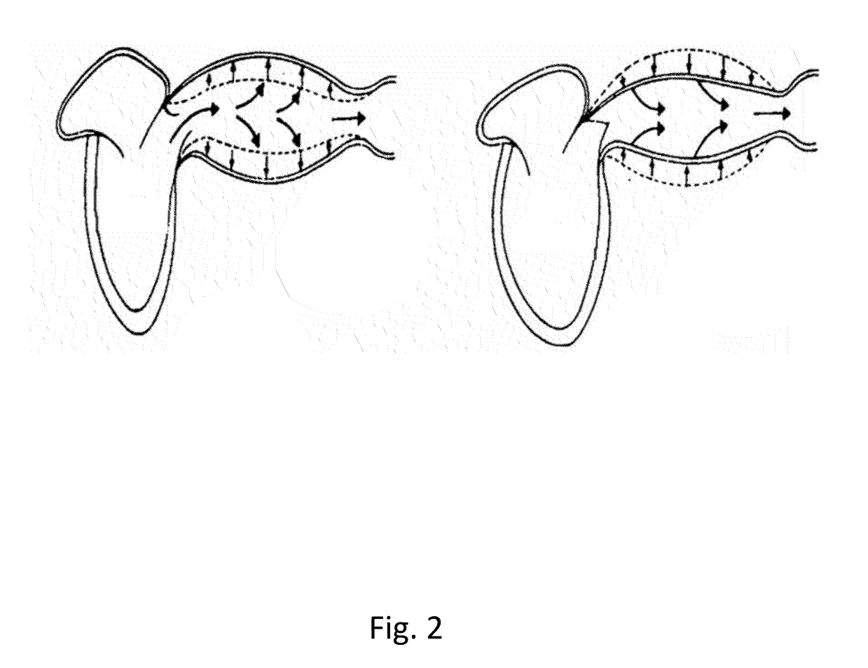

In “Radial pulse transit time is an index of arterial stiffness”, Zhang, Yong-Liang et al., Hypertens Res, 2011, Volume 34, Number 7, the authors use pulse pressure data obtained by an applanation tonometer based system and process the data to determine the arrive of the first and second systolic peaks. The resulting time difference or Pulse Transit Time (PTT) is used to create a time difference that is correlated with age, see FIG. 2. FIG. 2 is an illustration of the genesis of pressure pulses: after the opening of the aortic valve the pulse propagates through the aorta exchanging energy between the aortic wall and the blood flow. The authors infer a general trend between arterial stiffness and age and show a correlation between the time difference and age. The method is limited to only a peak detection method and no direct measure of arterial stiffness is presented and no association with diabetes is articulated.

The present invention can address the limitations of the prior art with a focus on diabetes assessment and the effective use of time separation, pulse amplitude and the shape of the pulse wave. The present invention is not constrained by historical limitations that compromise the degree of fit, make assumptions regarding curve shape (for example Gaussian) or use only peak separation metrics. These limitations limit the information that can be obtained for the pulse wave by ignoring higher order harmonics or simply by failing to use effective curve parameterization techniques.

The following example embodiment of the present invention addresses these limitations by effectively using more of the information available in the pulse wave. The approach is described as follows. Retrieve photoplethysmograph signals with methods described elsewhere in this description. Signals are then decomposed by mean of Discrete Wavelet Transform (DWT). The decomposition is not limited to DWT and embodiments can use other types of discrete and continuous type of wavelet decompositions (including but not limited to Daubechies wavelets, Hermitian wavelets, etc.). Wavelet real and complex—if present—valued coefficients are extracted to compose a vector of features. One vector of features will be generated for each case (patient, subject). Feature vectors are augmented by use of other clinical parameters including as examples quantitative and qualitative traits (including but not limited to systolic blood pressure, diastolic blood pressure, age, gender, etc). The resulting vector of features is then processed or evaluated by a classifier developed by one or more machine learning/pattern recognition approaches (including, but not limited to, Support Vector Machines, Random Forest, Decision Trees or any type of classifying trees, Clustering, Bayesian Networks, Neural Networks, etc). The output of the classifier defines a metric associated with diabetes state or an assessment of diabetes.

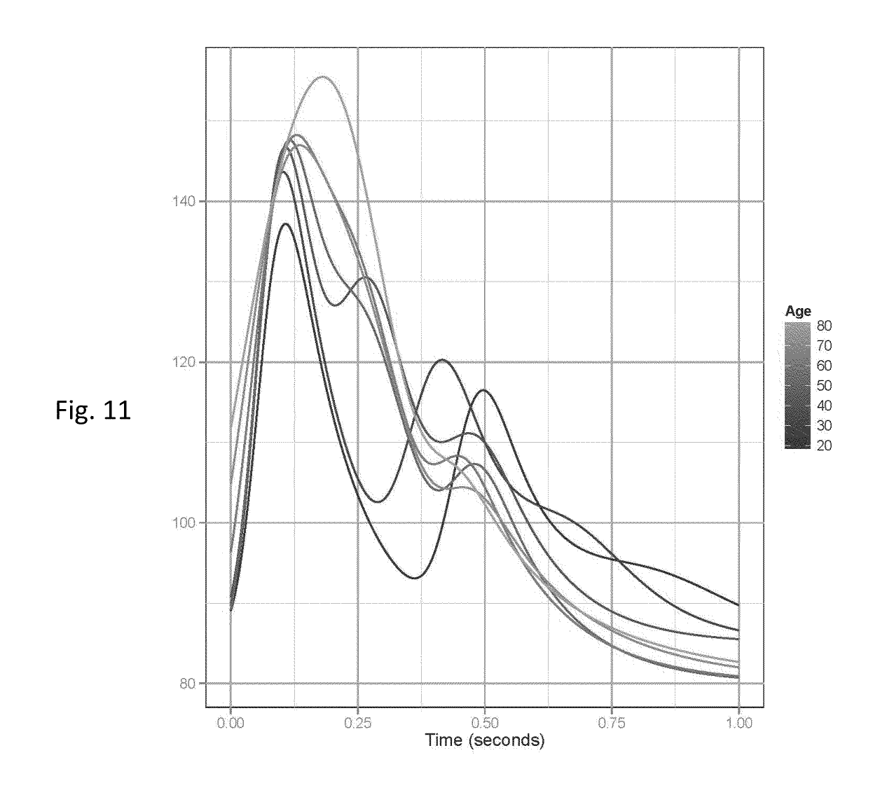

To demonstrate the superiority of the method, a set of representative physiological data has been analyzed according to the approach described in this invention. 2,000 photoplethysmographs from subjects at various stage of Diabetes Mellitus (but without other concomitant co-morbidites) and 2,000 photoplethysmographs from otherwise similar healthy subjects have been decomposed using DWT. FIG. 11 shows an example of the pulses used for analysis. The data represents a complex and confusing array of pulses with waveform differences. Clinically augmented vectors of wavelet features have been used to train a Support Vector Machine classifier. Cases were randomly assigned to the training and test sets. The classifier was trained using the training set only. The test or validation data was then classified by the Support Vector Machine classifier and the performance was accessed by Receiver Operation Characteristic (ROC) curve. As a control and to compare the goodness of the improved approach, Pulse Transit Time (PTT) as used by Zhang, Yong-Liang et al. was computed on of the very same dataset.

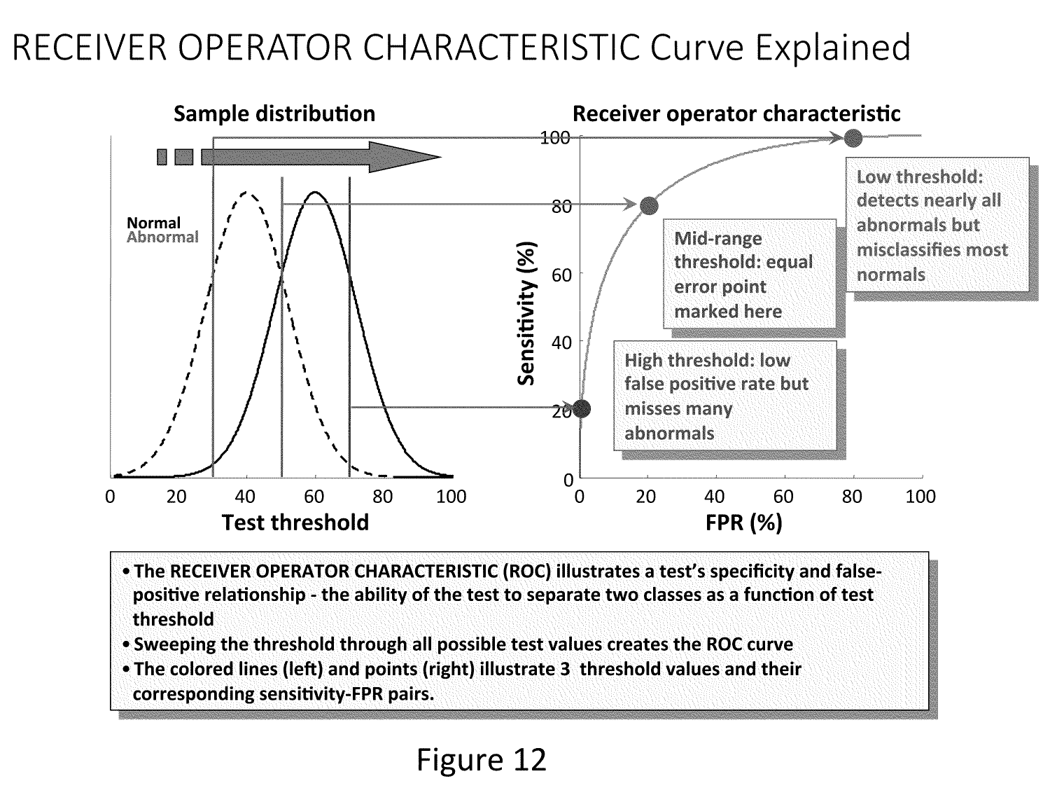

The Receiver Operator Characteristic (ROC) curve FIG. 12 illustrates a test's specificity and false-positive relationship or the fundamental ability of the test to separate two classes as a function of test threshold. The receiver operator characteristic (ROC) illustrates a test's specificity and false-positive relationship—the ability of the test to separate two classes as a function of test threshold. Sweeping the threshold through all possible test values creates the ROC curve. The colored lines (left) and points (right) illustrate 3 threshold values and their corresponding sensitivity-FPR pairs.

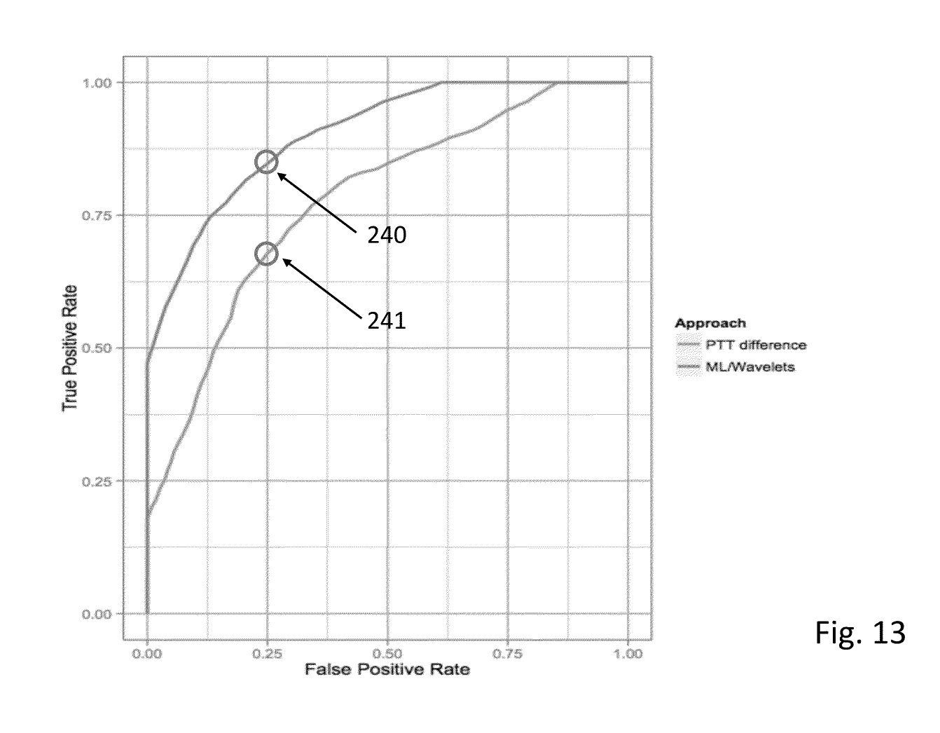

Examination of FIG. 13 shows the significant performance improvement possible. The Area Under Curve (AUC) for the Wavelet approach is greater than the AUC for the PTT approach and the test performance is better are every point on the ROC curve. The performance with a false positive rate of 25% is a sensitivity of 85% (240) for the Wavelet approach in comparison to a 68% (241) for the PPT approach. The improved processing method yields a 25% increase in sensitivity. The Machine Learning with Wavelet feature decomposition approach proves to be superior in diagnostic power, to the PTT based approach.

Those of skill on the art will appreciate that other decomposition methods can be suitable, such as principle component analysis (PCA), semi-supervised local fisher discriminant (SELF), independent component analysis (ICA) and neighborhood component analysis (NCA). The resulting decomposition data can then be processed to determine the best maps between normal and abnormal samples based upon Euclidean distance metrics. A method for learning the best distance metric is structure preserving metric learning (SPML) methods. Methods that utilize the true information content of the pulse wave can be preferred over those with defined limitations or assumptions regarding the structure of the data.

Improved Pulse Photoplethysmogram Measurement System by Cross Polarization.

Photoplethysmography (PPG) is an optical measurement technique that can be used to detect blood volume changes in the microvascular bed of tissue. The basic form of PPG technology requires only a light source to illuminate the tissue (e.g. skin), and a photodetector to measure the small variations in light intensity associated with changes in perfusion of the tissue under examination. PPG is most often employed non-invasively and operates at a red or a near infrared wavelength. The most recognized waveform feature is the peripheral pulse, and it is synchronized to each heartbeat. Despite its simplicity the origins of the different components of the PPG signal are still not fully understood. It is generally accepted, however, that they can provide valuable information about the cardiovascular system.

The simple Lambert-Beer Law derivation of pulse oximetry assumes a single, well-defined light path common to each of the wavelengths that pass through the tissue. In this simplistic overview, all photons travel the same path, which is defined by the physical separation between the source and the detector. However, in reality all wavelengths of light are strongly scattered by human tissue; the detected light is more accurately described as an ensemble of independent photon paths (Patterson M, Chance B, Wilson B. Time resolved reflectance and transmittance for the non-invasive measurement of tissue opti [symbol] cal properties. Appl Opt 1989; 28:2331-6, Bonner R, Nossal R, Havlin S, Weiss G. Model for photon migration in turbid biological media. J Opt Soc Am A 1987; 4:423-32 7,8). Some of the detected photons travel shorter routes without migrating far from the direct line between the emitter and detector, and some scatter farther from this line without being absorbed or lost at a boundary (photons that are absorbed or lost cannot be measured by the photodetector). The longer-traveled photon routes provide more interaction with blood, and the incremental difference between systolic and diastolic concentrations of tissue blood creates a greater impact on the relative number of survivors (greater detected pulse amplitude). Conversely, detected photons traversing the shorter distances are less exposed to the cycling blood level and survive with a more uniform likelihood between systolic and diastolic conditions (smaller detected pulse amplitude).

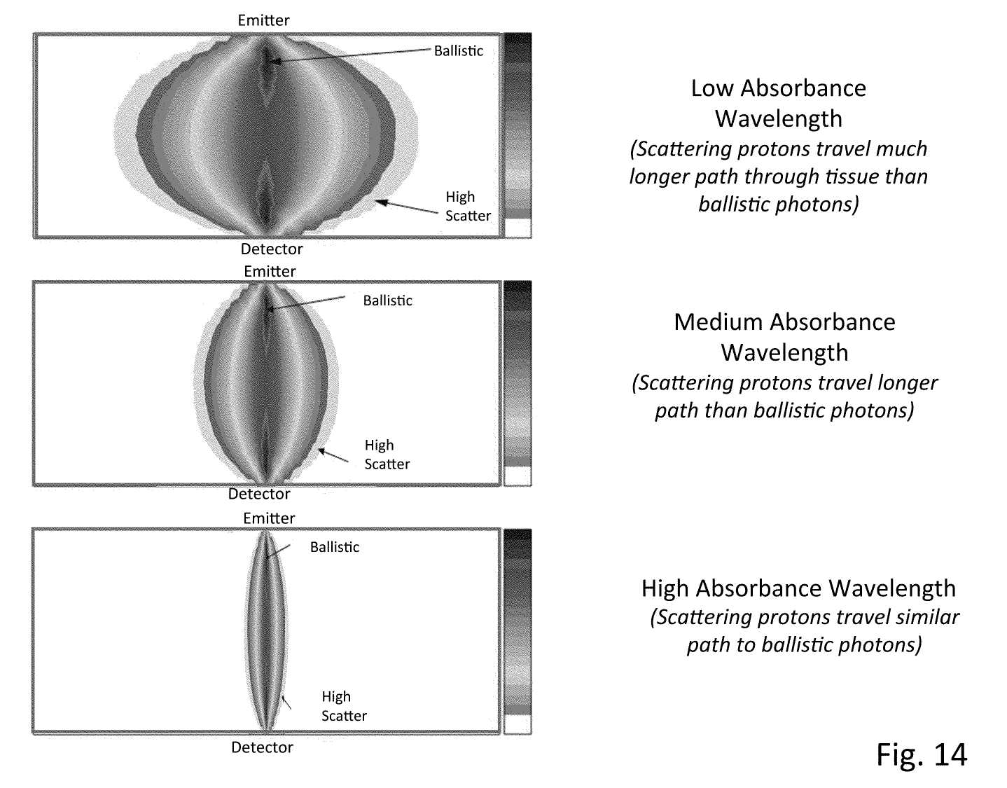

As the absorption of the light increases, detected light tends to come from the shorter paths. FIG. 14 illustrates this graphically using the results of a random walk model for photons scattering through tissue media. Those photons traveling directly or almost directly from the emitter to the detector are referred to as “ballistic” photons. These photons nominally travel a more direct path between the emitter and detector but will in most cases encountered some degree of scattering interaction. The “high scattering” photons travel a significantly different trajectory and in fact have a total optical path length that is significantly greater than the path length traveled by the ballistic photons.

As the absorbance at a given wavelength increases, the probability of the photon becoming absorbed increases. As the probability for a photon becoming absorbed at any given step increases (higher absorption), fewer surviving routes reach the periphery or survive a highly scattering trajectory through the tissue. Detailed examination of FIG. 14 shows that for a low absorbance sample, many photons are able to travel into the periphery and be subsequently detected by the photodetector. As the absorbance for a given wavelength increases, the number of photons able to travel the additional distance due to scattering decreases. Thus, the photons that are detected have traveled (on average) a more direct pathway. With higher absorption as shown in the bottom graph of FIG. 14, the average path length difference between the ballistic photons in the scattering photons decreases. In very simple terms, increasing absorbance decreases the overall path traveled as well as the variance observed by individual photons.

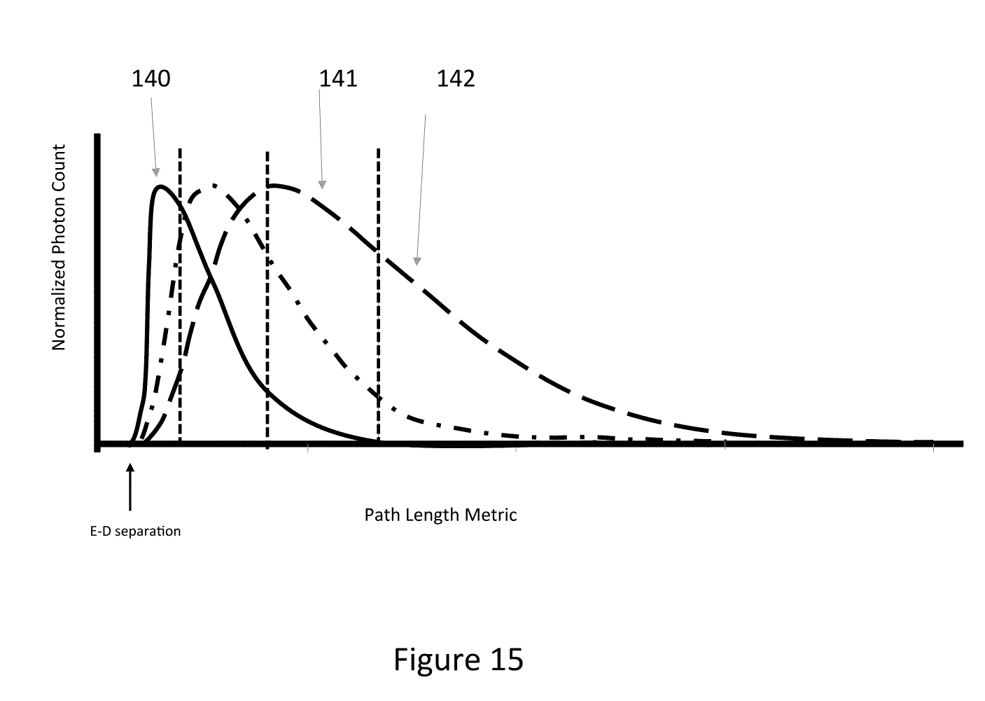

FIG. 15 provides the estimated path length distributions for the three scenarios shown in FIG. 14. The final detected signal comprises a mixture of all surviving photon paths. FIG. 15 shows relative path length distributions for a normalized set of detected photons for the low absorbance (142), middle absorbance (141) and high absorbance conditions (140). Each photon's path length is effectively a product of the number of steps taken in its random walk to the detector and their step size. Notice that as the absorption increases, not only are fewer total photons detected, but the distribution shifts toward the shorter routes. The vertical dotted line in each curve indicates their respective mean path lengths (I) values that are several times greater than the physical separation between the emitter and photodetector (E-D separation). The number of surviving photons that travel the minimum distance directly across the slab without being scattered away from the straight line is small; path lengths less than the E-D separation are impossible.

This rather nuanced understanding of the interaction between absorption, scatter, and overall pathlength can be used for procurement of an improved PPG signal. As stated above, the longer-traveled photon routes provide more interaction with blood, and the incremental difference between systolic and diastolic concentrations of tissue blood creates a greater impact on the relative number of survivors (greater detected pulse amplitude). Therefore, the preferential selection of only those photons that have traveled longer distances will lead to an improved signal.

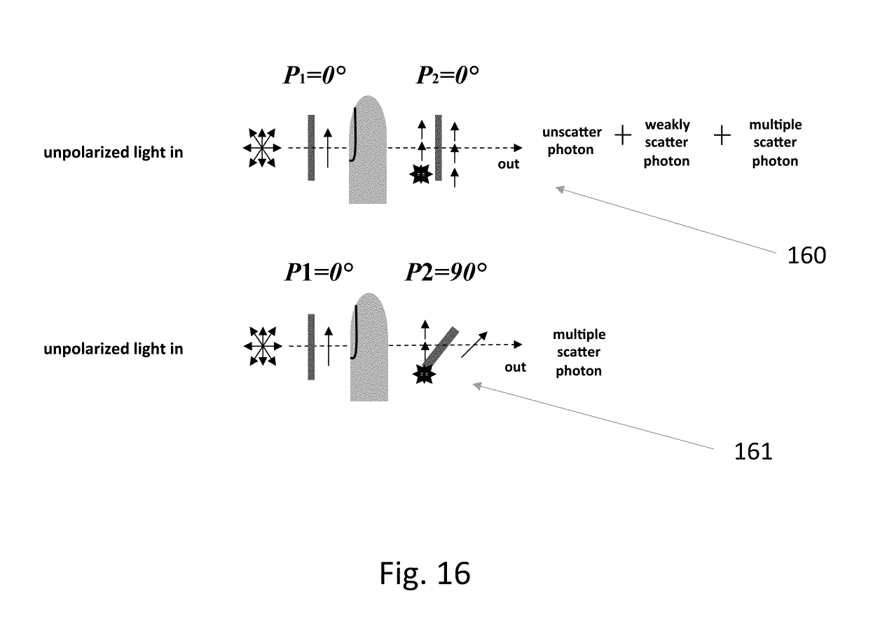

The system shown in FIG. 16 uses an optical sampling method based upon cross polarization to preferentially select those photons that have been scattered by the tissue. When polarized light illuminates a scattering medium, weakly scattered light will emerge in its original polarization state, while multiply scattered light will emerge with random polarization. Element 160 shows a parallel polarization method, while 161 shows cross polarization sampling of the finger. Such a cross polarization sampling system can be used to preferentially reduce those photons that have traveled directly or almost directly through the tissue such that their polarization state has not been altered. In such a system, the illumination light is passed through a polarizer and defines an illumination orientation. The collected light (on the opposite side of sample) can then be passed through a second polarizer. The orientation difference between the illumination polarizer and the collection polarizer determines what type of light is preferentially selected. A parallel, or zero degree difference, between the illumination and collection polarizers emphasizes light that has maintained the same polarization state and traveled through the tissue with a minimum number of scattering events. This type of arrangement is referred to as a parallel polarization system (160). For the procurement of photons that have maximally interacted with blood, a cross polarization system is utilized (161). In the cross polarization system, the photons have had their polarization state randomized due to multiple scattering events and have therefore traveled a greater distance. An optical sampling system that preferentially selects for highly scattering photons will have better sensitivity to volume changes in will result in an improved PPG measurement.

Improved Pulse Photoplethysmogram Signal by Use of Both Types of Optical Information.

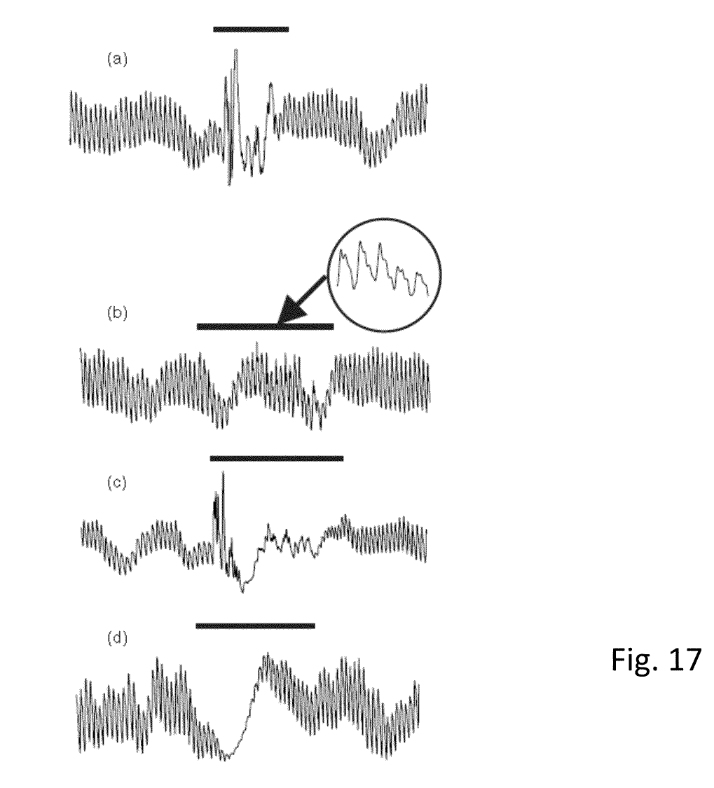

Pulse photoplethysmogram (PPG) measurements are sensitive to small volume changes in the distal finger. These changes are remarkably sensitive to slight changes in pressure of the finger under examination and any change that influences the volume change in the distal extremity. It is recognized that PPG measurements are sensitive to breathing, intrathoracic pressure, changes in autonomic tone, venous return from other parts of the body, left ventricular return, and any sort of motion. It is well established that PPG measurements are quite sensitive to patient and/or probe—tissue movement artifact. FIG. 17 shows examples of the types of measurement artifacts and the extremes in physiological variation that can be seen in PPG recordings. Each recording is from the index finger site over a period of 1 minute and the artifact/physiological events are marked. (a) An episode of gross movement artifact or PPG probe cable tugging lasting approximately 15 seconds. (b) Hand or finger tremor, (c) a bout of coughing, and (d) marked changes in the breathing pattern (a deep gasp or yawn). These types of artifacts and physiological variations add noise to the overall measurement and add significant complexity to subsequent wave analysis. The automatic detection of such motion artifacts, and separation of those from good quality although highly variable pulse recordings, is a non-trivial exercise in computer signal processing. Computer-based filtering, feature extraction and waveform averaging have also been employed in PPG pulse wave analysis, including the analysis of frequency (de Trafford et al 1982, Okada et al. 1986, Nitzan et al. 1994, Bernardi et al. 1996, Grohmann et al. 1996a, 1996b, Larsen et al 1997, Sherebrin and Sherebrin 1990), joint-time frequency (Yan et al. 2005), artificial neural network (Allen and Murray 1993, 1995, 1996, 1999, Weng et al 1998), systems identification and transfer function modelling (Cohn et al. 1995, Allen and Murray 1993, 1995, 1996, McVeigh et al. 1999, Millasseau et al. 2000), principal component analysis (Enríquez et al. 2002), nonlinear and chaos theory (Christ et al. 1997, Bhattacharya et al. 2001), cross correlation (Allen and Murray 2000a, Drinnan et al. 2001) and double differentiation (acceleration plethysmogram, Takada et al. (1996-97), Takazawa et al. (1998), Bortlotto et al (2000)).

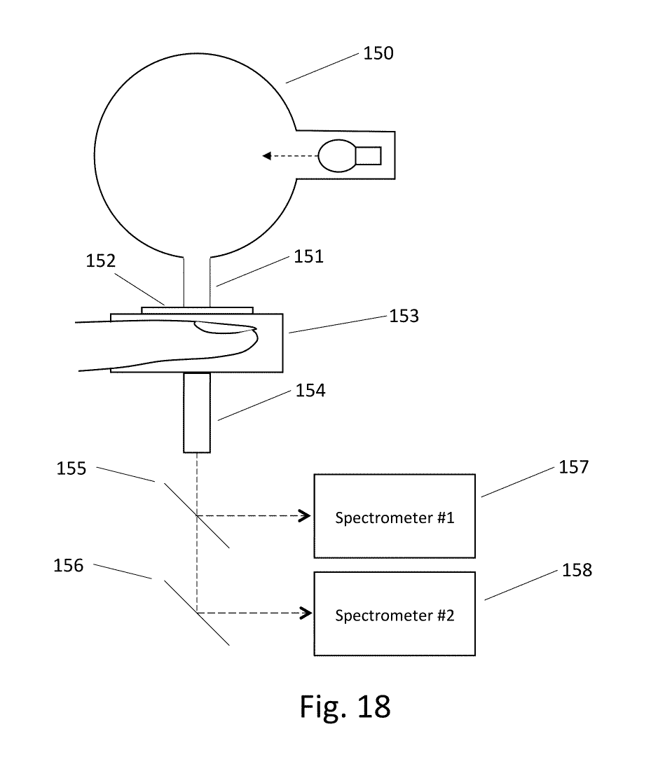

A dramatic improvement in overall signal quality and immunity to motion artifacts can be obtained by effectively capturing cross polarization and parallel polarization photons concurrently. FIG. 18 shows an example embodiment with a multi-wavelength source and integrating device 150, shown as an integrating sphere, illuminating the tissue with an optical coupling system 151, shown as a light pipe. The illumination light is transmitted through polarizer 152 to define a polarization state. Following polarization, the photons enter the tissue and are absorbed and scatter by the finger, which is held steady by a finger support system 153. The light exiting the tissue is transferred to a polarizing beam splitter 155 by an optical system 154, shown as a light pipe. The polarizing beam splitter 155 separates the light into parallel and perpendicular components. A given polarization of light is then measured on spectrometer #1 157 and the other polarization state on spectrometer #2 158. The system enables the simultaneous acquisition of both parallel and perpendicular polarized light at multiple wavelengths through the tissue.

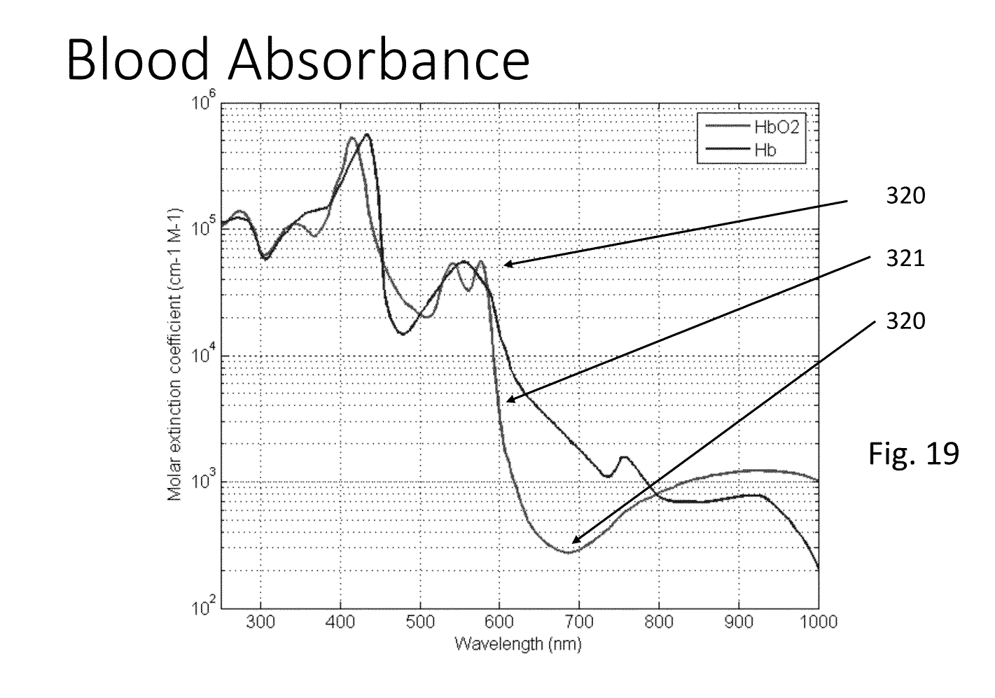

The data obtained can be processed in a manner that facilitates the procurement of a high signal to noise PPG with compensation for motion artifacts. FIG. 19 shows the absorbance characteristics of blood. The absorbance differences between blood over this wavelength range can be leveraged to create and improved PPG signal.

As background, the absorption coefficient (μₐ) is defined as the probability of photon absorption in tissue per unit path length. Different tissue components have different (μₐ) values. Moreover, (μₐ) is a function of wavelength. The molar extinction coefficient (ε) the term plotted on FIG. 19 is another parameter that is used to describe photon absorption in tissue. By multiplying e by the molar concentration and by In(10), one can convert ε, to (μₐ).

Blood consists of two different types of hemoglobin: oxyhemoglobin (HbO₂) is bound to oxygen, while deoxyhemoglobin (Hb) is unbound to oxygen. These two different types of hemoglobin exhibit different absorption spectra that are normally represented in terms of molar extinction coefficients, as shown in FIG. 19. The molar extinction coefficient of Hb has its highest absorption peak at 420 nm and a second peak at 580 nm. Its spectrum then gradually decreases as light wavelength increases. On the other hand, HbO₂ shows its highest absorption peak at 410 nm, and two secondary peaks at 550 nm and 600 nm. As light wavelengths passes 600 nm, HbO₂ absorption decays much faster than Hb absorption. The points where the molar extinction coefficient spectra of Hb and HbO₂ intersect are called isosbestic points. Most oximeters operate with two wavelengths, a red wavelength of around 660 nm and an infrared wavelength of 900 nm.

The present invention enables the measurement of spectra in both parallel (Sₚₐᵣ) and perpendicular (Sₚₑᵣ)) polarizations states such that the degree of polarization (DOP) can be calculated. Degree of polarization (DOP) is a quantity used to describe the portion of light which is polarized. The DOP of the light passing through a turbid medium is defined as:

$$D O P = \frac{{I}_{II} - {I}_{⊥}}{{I}_{II} + {I}_{⊥}}$$

$$D O P = \{ \begin{matrix}1 & represents linearly polarized light {I}_{II} >> {I}_{⊥} \\ 0 & represents totally depolarized light {I}_{⊥} \to {I}_{II}\end{matrix}$$

For purposes of explanation, consider the three wavelength shown in FIG. 19, high absorbing wavelength 320 at 580 nm, medium absorbing wavelength 321 at 600 nm and a low absorbing wavelength 320 at 700 nm. The degree of polarization can be calculated for these three wavelengths. For high absorbance wavelengths, the DOP will be approach 1 since photons traveling longer distances will have been absorbed. At low absorbance values, the DOP will be less than 1 due to the contribution of the scattering photons. Stated differently, at low absorbance the scattering photons contribute at a significant level relative to the parallel photons resulting in DOP values less than 1.

As stated above, motion, pressure, as well as changes in right heart filling can increase or decrease the overall blood flow to the peripheral tissue. Thus, an effective PPG measurement system should compensate for these changes while maintaining a high fidelity PPG signal. The use of a time varying DOP signal creates a method for motion artifact compensation/normalization. Specifically, photons measured from those areas of the spectrum with high absorbance will contain photons via parallel and perpendicular polarization that have nominally traveled similar distances through the finger, see FIG. 14. The photons measured at wavelengths with lower absorbance will contain photons via parallel and perpendicular polarization that have nominally traveled different distances through the finger, see FIG. 15. As stated above, those areas or wavelengths of the spectrum with low absorbance will be more sensitive to blood/scatter changes

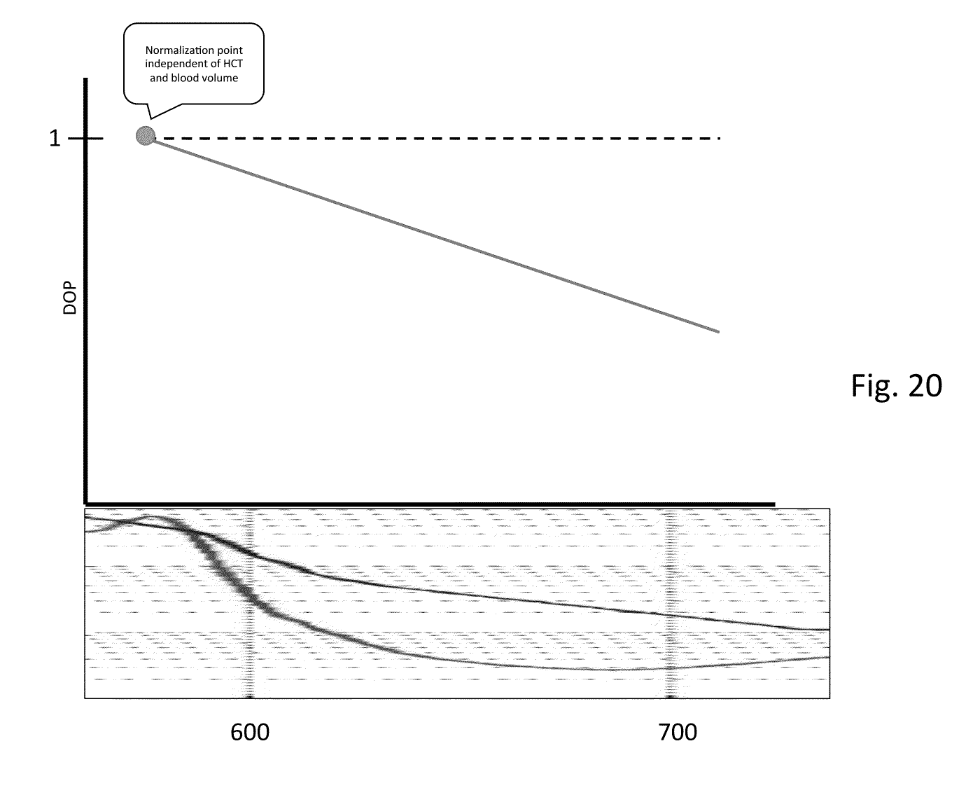

The different absorbance characteristic can be used through the DOP calculation to create a multi-wavelength DOP measure. FIG. 20 shows a pictorial representation of DOP over the wavelength range of 580 to 700 nm. As the absorbance at a given wavelength decreases, the DOP decreases, as shown by the sloped line in FIG. 20.

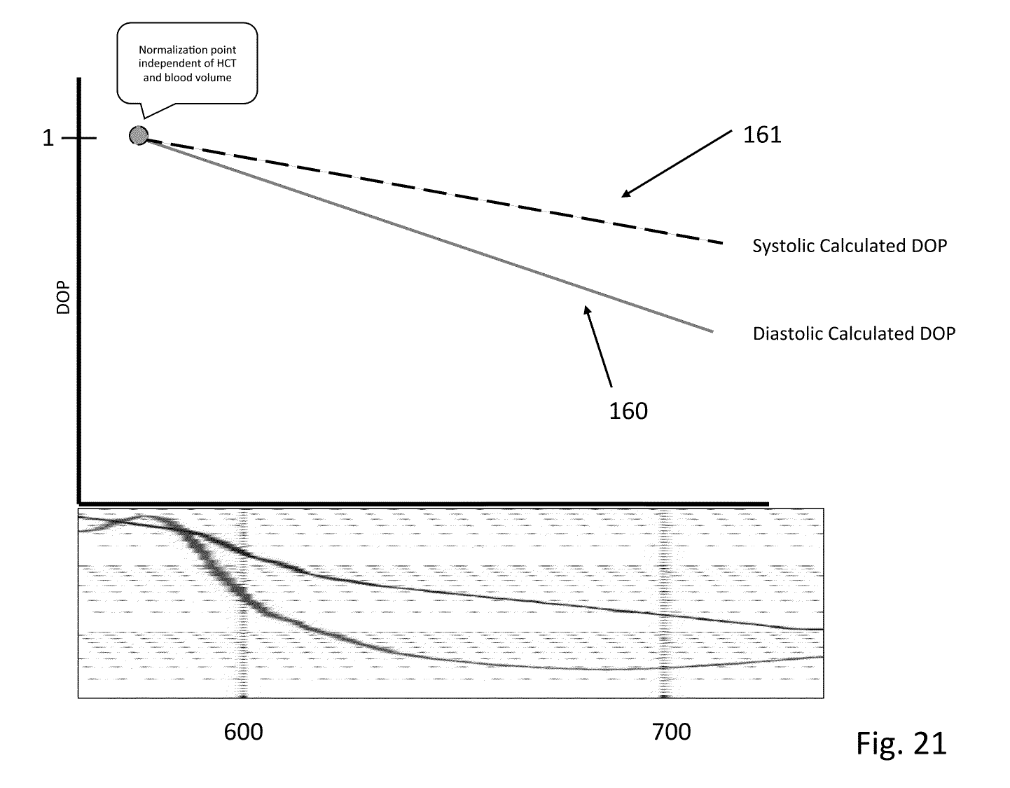

A key observation is that the slope of the DOP is dependent on the absorbance of the sample. Changes in blood volume will causes changes in absorbance and therefore changes in the slope of the DOP. In diastole the blood volume is lower that systole, resulting in less blood absorbance. Less absorbance creates lower DOP values at the same corresponding wavelength 160 in FIG. 21. During systole the volume of blood is increased, resulting in DOP values that are increased at their corresponding wavelengths 161, FIG. 21 due to the increased absorbance of the finger. The result of this process is a multi-wavelength DOP measurement that is self-normalized at the high absorbance wavelength but maintains excellent sensitivity to blood volume changes through changes in slope.

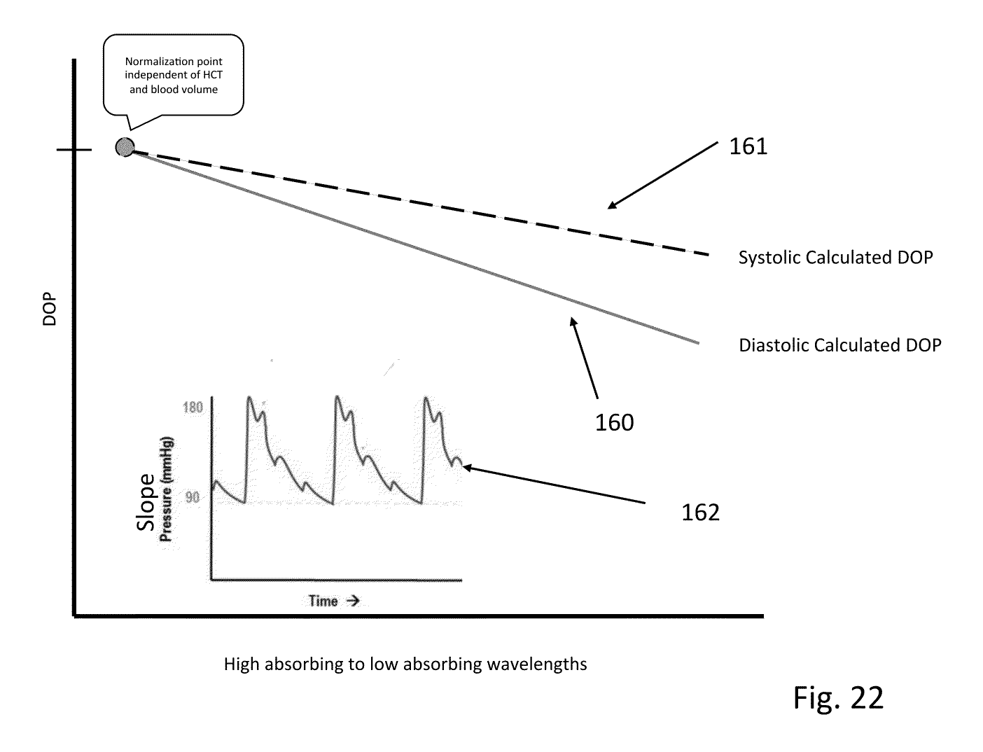

FIG. 22 expands the concept to all wavelengths where the x-axis is a sorted wavelength axis from the highest to lowest absorbing wavelength. The effective slope of the DOP plot can then be used to account for changes in blood volume and create a signal containing PPG information 162. It is important to point out that the signal in 162 contains information associated with blood volume changes much like a conventional PPG but the information is derived through a superior process. The system and method uses polarization for photon selection, leveraging wavelength absorbance differences and the degree of polarization calculation to create a superior PPG signal. Other algorithms or processing methods could be used that result in metrics that mimic the information for the DOP. Thus, the DOP should be considered as an example method that uses different sources of light to create an improved PPG signal.

Improved Pulse Photoplethysmogram System by Use of Numerical Aperture.

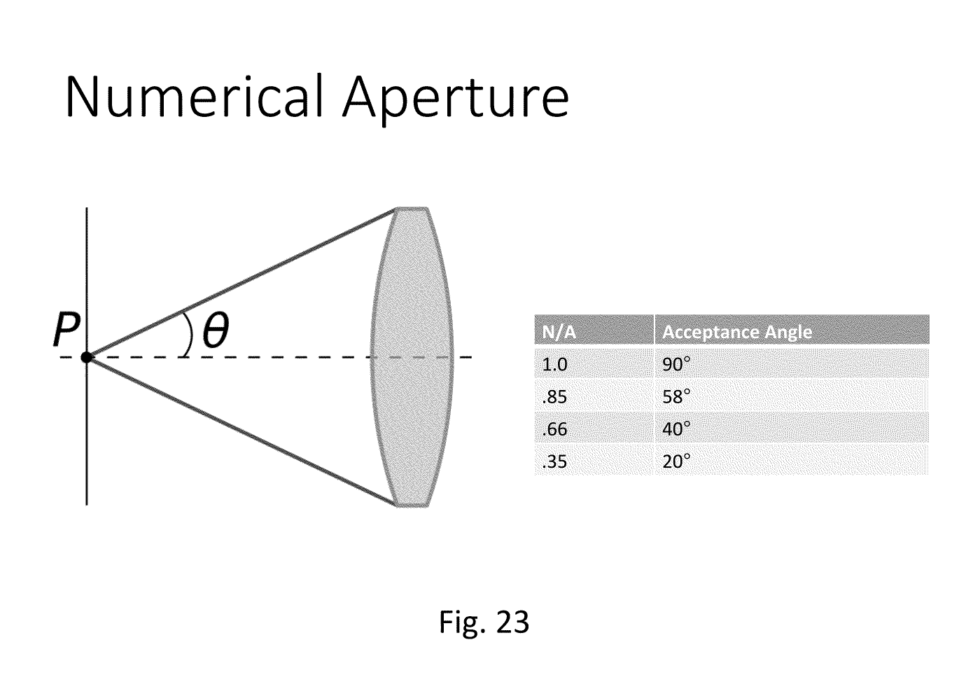

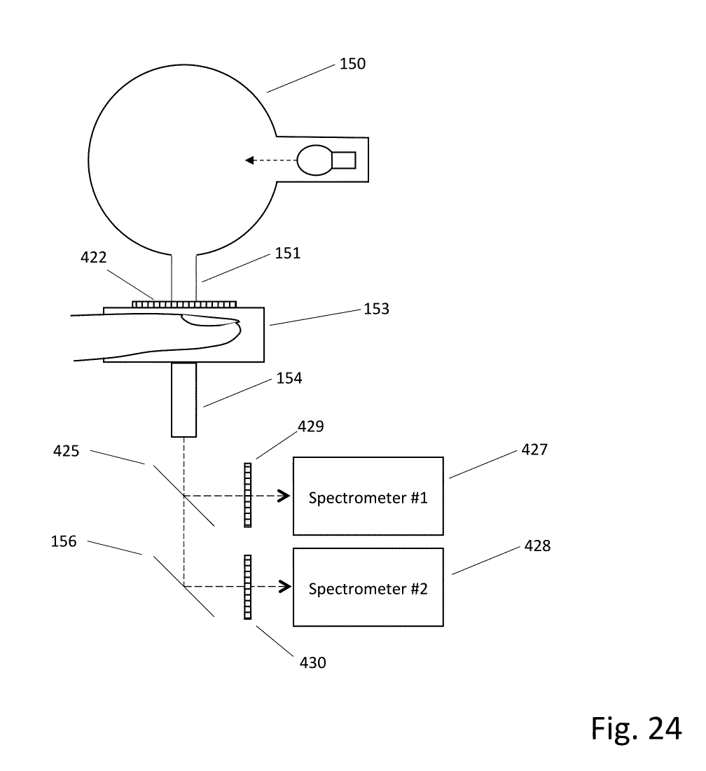

Cross polarization is one method for selecting photons that have traveled though tissue with a defined scatter or path characteristic. The effective use of numerical aperture can also be used to obtain similar or related results. In optics, the numerical aperture (NA) of an optical system is a dimensionless number that characterizes the range of angles over which the system can accept or emit light, see FIG. 23. Re-examination of FIG. 14 shows that photons that have effectively traveled straight thought the tissue or with minimal deviation (aka ballistic photons) could be accepted by a system with a small numerical aperture. A larger numerical aperture collects photons with a greater range of angles. FIG. 24 shows a reconfiguration of the system of FIG. 18 but where the polarization aspects of the system have been replaced by methods for numerical aperture control. As shown in FIG. 24, fiber optic face plates (FOFP) are used for numerical aperture control. FOFP are can be used for numerical aperture control, are small in size and the desired NA can be selected from multiple options. Numerical aperture control can be obtained through multiple means, and can also include standard lens or imaging systems. As shown in FIG. 24, FOFP 422 is used to condition the light entering the tissue. The light exiting the tissue is transmitted to beam splitter 425. This beam splitter can be a 50/50 splitter or can be designed to normalize photon count by sending a higher percentage of light to the low NA portion of the system. The light is then transmitted though the low NA FOFP 429 for detection on spectrometer #1 427. The remaining light is sent through the higher NA FOFP 430 and collected by spectrometer #2 428.

The system effectively allows the capture of data that has predominantly ballistic travel characteristics via the data recorded on low NA spectrometer 427. Concurrently, photons that may be highly scattered or ballistic are captured on the high NA spectrometer 428. The separation of the data into high scattering photons and more ballistic photons can occur by subtraction of the high NA data from the low NA data:

Low NA data=ballistic photons+weakly scatter photons+photons with final trajectory satisfying NA

High NA data=low NA photons+multiple scatter photons+photons satisfying large NA

Highly scattering photons=High NA−Low NA

Operation of System.

In practice, the present invention creates a method and system for diabetes assessment that is remarkably easy to use. The test does not have a fasting requirement, as the test is not a direct assessment of point in time glucose maintenance. In contrast, the measurement effectively integrates the damage due to slight variations in glucose as well as the early manifestations of diabetes. Therefore, any individual can be tested at any time without pretest compliance issues. The individual under examination can simply place their finger in the test system for a period of time to enable device to procure one or more observations of the cardiac cycle. If ancillary information is needed, this information can be entered into the device for effective utilization. The system can process the optical information in combination with any ancillary information provided, to provide a real-time result. The ability to provide test results at the time of testing is very valuable. A diabetes risk score can be provided to the patient. The numerical value of the risk score can provide the individual with information regarding their probability of having prediabetes or diabetes. Additionally, the device can provide a more direct assessment by simply defining an individual state as normal, pre-diabetic, or diabetic. The device can also, or as the alternative, provide an “arterial age” measurement. Such measurement can simply state that the information obtained from the patient is most consistent with a given age profile. Accelerated aging would be viewed as problematic and additional medical work required.

Additional Optical Systems.

As one of skill in the art will recognize, there are multiple instrument variations that can be used for the collection of data that allows the above improved PPG signal to be created. It is also important to note that the calculation of Aortic Transit Time or other measures of arterial stiffness can be obtained from a simple conventional oximeter. In fact, these parameters can be calculated from a single LED system. Therefore, multiple optical systems or instrument designs can be used to assess arterial stiffness for the determination of diabetes assessment.

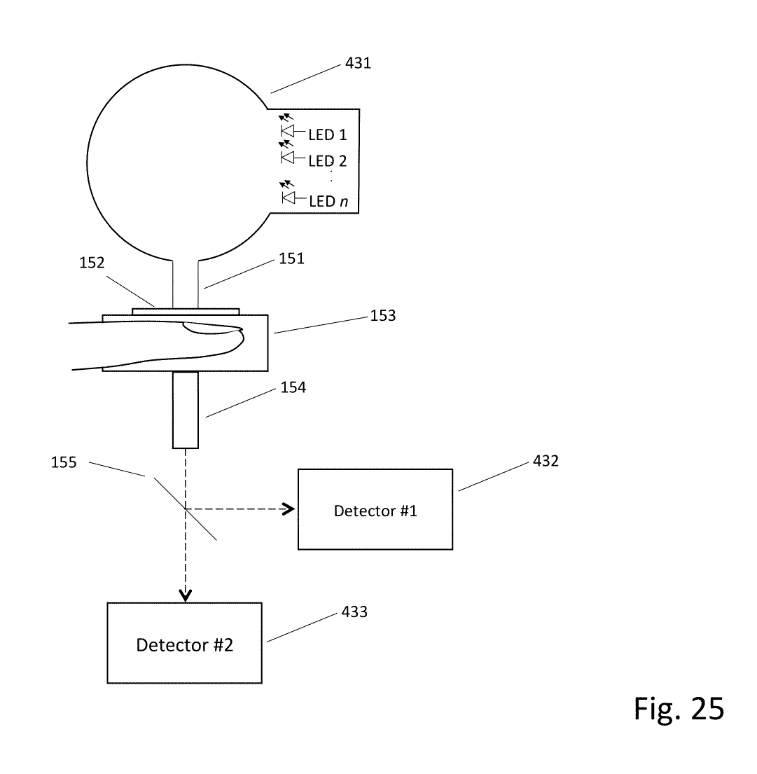

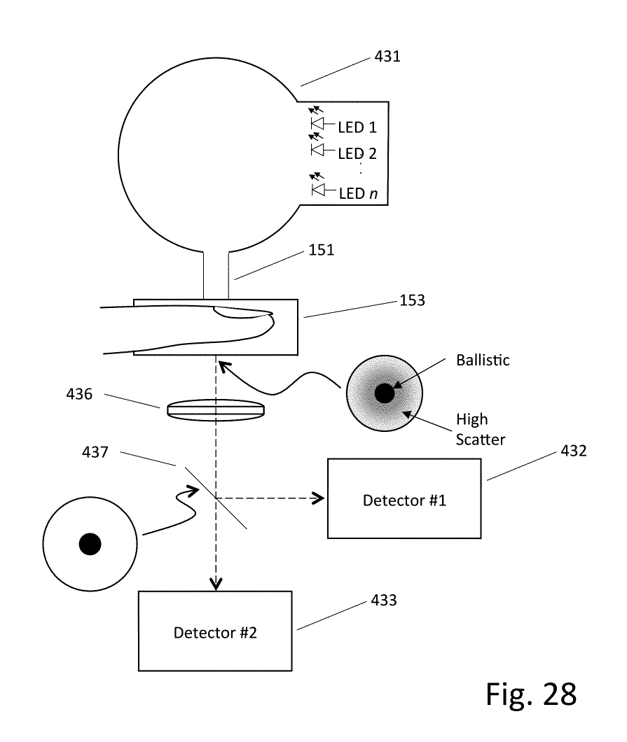

The optical systems shown in FIGS. 15 and 24 should be viewed as illustrative examples. These examples depict the use of a continuous wavelength spectrum and the concurrent recording of multi-wavelength data. As one of ordinary skill in the art would appreciate, a multi-wavelength source can be replaced by discrete wavelengths; various multiplexing schemes can be utilized such that only one detector system is needed; and only a few discrete wavelengths might be required for analysis. Additional optical configurations are shown in FIGS. 25, 26, 28 and 29. Additionally, a single detector system can also be used.

For example, FIG. 25 depicts an example embodiment having one or more light emitting diode (LED) sources 431 and the light collected from the finger after reflecting and transmitting through a polarizing beam splitter 155 is received by a first 432 and second 433 detector.

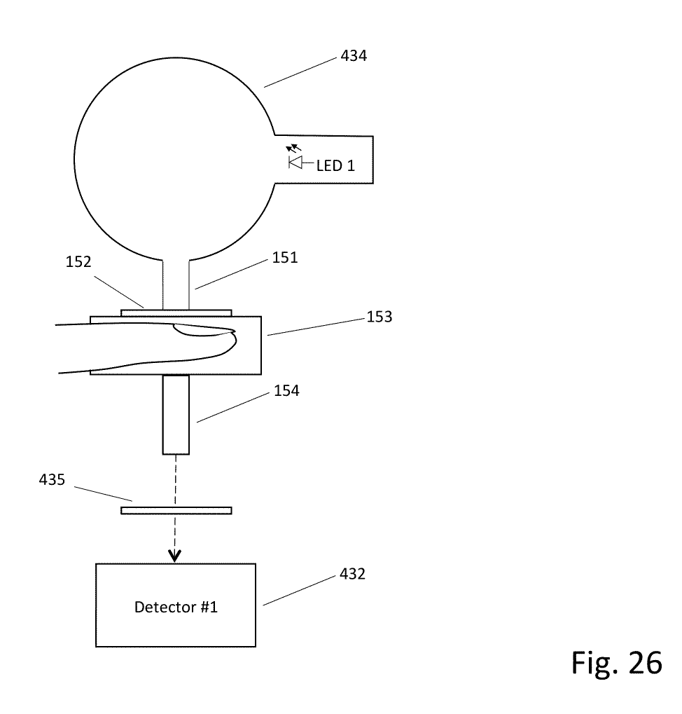

In another example embodiment, FIG. 26 shows a further simplification having one LED source 434 and a single crossed polarizer 435 before a detector 432.

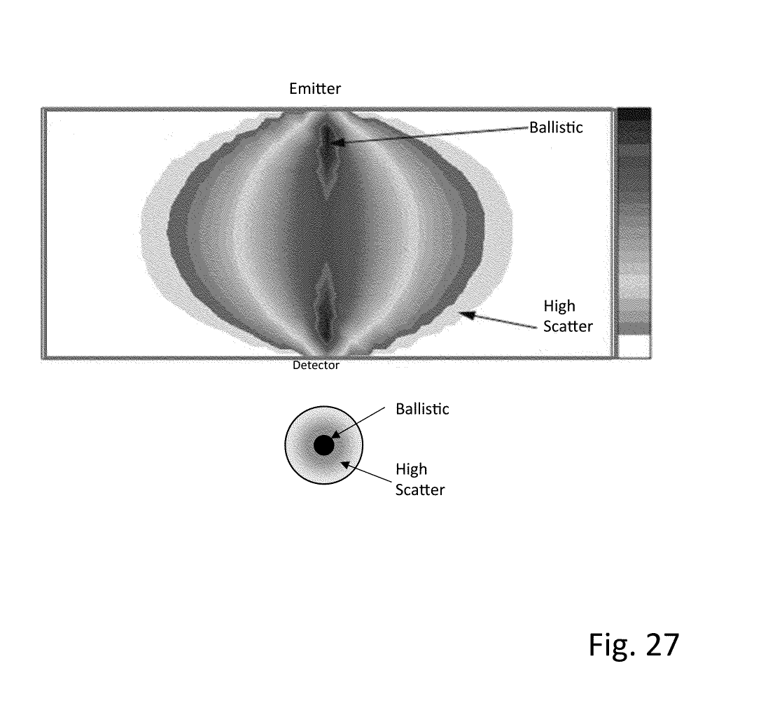

Referencing FIG. 11, a portion of which is depicted in FIG. 27, it can be seen that ballistic, or very low scattered, photons propagate through and exit the tissue very near the optical axis (defined as the line between the emitter and detector). This area of ballistic photons is shown as the dark, central region. Highly scattered photons will result in a larger area of emission upon exiting the tissue. This property of spatial differentiation between ballistic and scattered photons can be exploited. FIG. 27 depicts an embodiment whereby a lens 436 is used to image this region onto a substrate 437 having a reflective area coincident with ballistic photons. The ballistic photons are reflected toward a first detector 432 and the scattered photons are transmitted to a second detector 433.

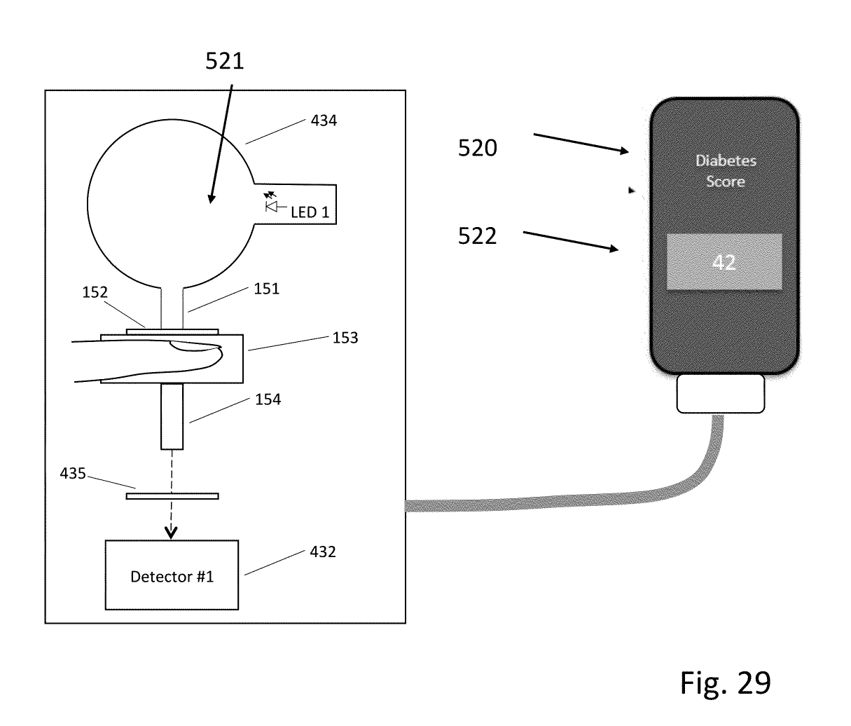

FIG. 29 shows a system comprising an optical measurement system 521, data acquisition system (e.g., a smart phone) 520 that records information and transmits data to central processing location (not shown) for analysis of the data. The central processing location processes data, determines the diabetes assessment score, and can manage invoicing and payment.

Additional Information.

Although the invention disclosure is written with a focus on diabetes assessment, it is important to note that other parameters can be assessed through pulse wave analysis using the improved data acquisition system as well as processing methods that can be applied to these measurements. Specific expansions or uses of the technology can include determination of hypertension, assessment of vascular graft failures, heart valve failure, and coarctation of the aorta. Additionally, the system described herein can also be used to assess for aortic aneurysms. Presence of an aortic aneurysm will dramatically influence the overall compliance of the aorta. Such an alteration in arterial stiffness can be observed with this system.

Of particular interest is the determination of intravascular volume status. Decisions regarding fluid therapy, whether in the operating theatre (OT), intensive care unit (ICU), emergency department (ED), dialysis center or general ward, are among the most challenging and important tasks that clinicians face on a daily basis. Specifically, clinicians would agree that both hypovolemia and volume overload increase the morbidity and mortality of patients. The determination of volume status is especially difficult due to the physiological response of the vascular system. Our homeostatic mechanisms have evolved (over thousands of years) to deal with hypovolemia (tachycardia, vasoconstriction, and blood flow redistribution). Thus, the body will maintain reasonable blood pressure until the capacity to deal with hypovolemia has been exhausted. As this juncture the patient will rapidly transition into hypovolemic shock, a life threatening condition. This transition often occurs without warning and tools to better predict hypovolemia are needed. Currently invasive catheters are placed in the central vasculature for volume assessment.

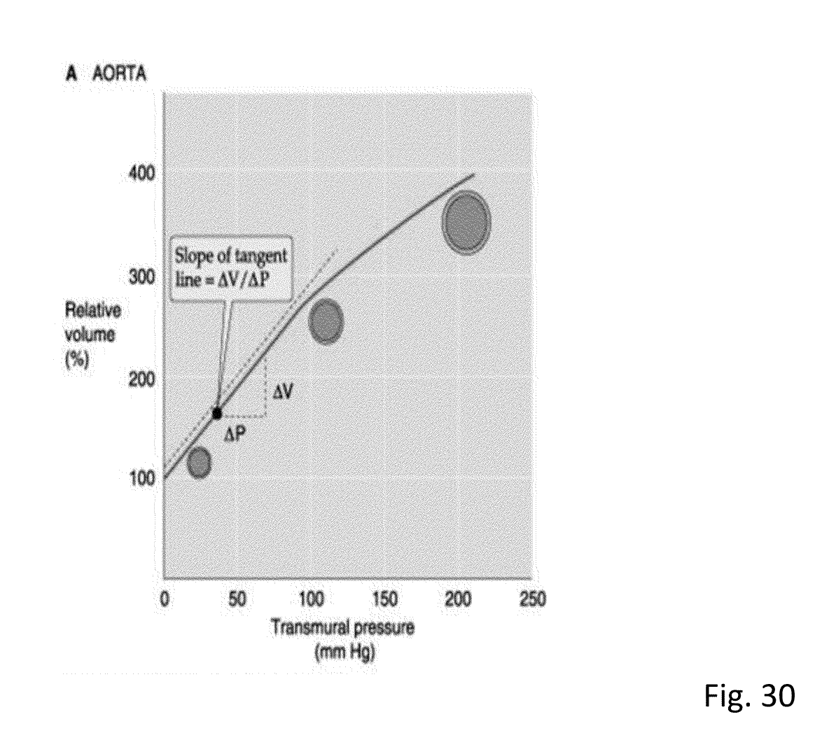

Intravascular volume status has a direct impact on the elastic state of the aorta. Thus, changes in vascular volume, especially at the extremes, impact arterial compliance and specifically the aortic transit time and pulse wave velocity in the aorta. As the volume status of the patient approaches hypovolemia the amount of change in aortic transit time will increase for a unit change in volume due to the nonlinear characteristics of the aorta. FIG. 30 shows the relationship between relative volume and transmural pressure.

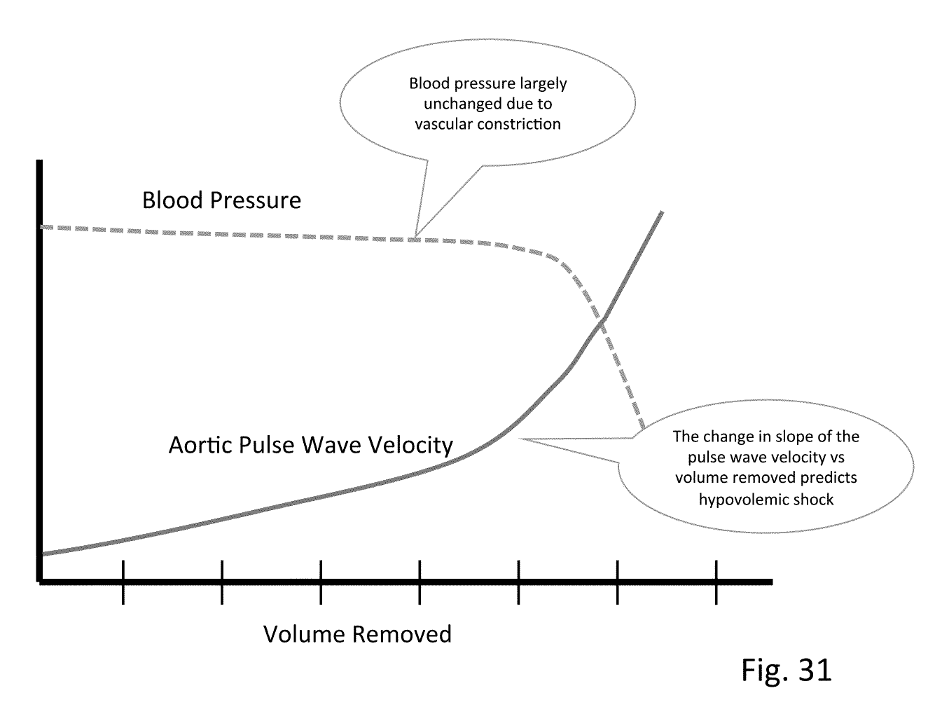

As it relates to aortic pulse wave velocity, the velocity of for a given person is dependent upon their aortic stiffness, as determined by age, diabetes status, hypertension, and end stage renal disease, and the volume status of the individual. As shown pictorially in FIG. 31, aortic pulse wave velocity and specifically the change in pulse wave velocity per unity change in volume status can serve as a metric for intravascular volume status and specifically for determination of impending hypovolemia. It is important to note that hypovolemia is associated with approximately a 20% loss of intravascular volume, something that is not present in a standard ambulatory population. The described system is focused on those volume assessment situations that occur in the emergency room, operating room, intensive care unit and dialysis clinic. As an individual becomes hypovolemic, the slope of the relationship between pulse wave velocity and a unit change in volume changes dramatically. The resulting slope can be used for volume assessment and to specifically assess when hypovolemia is likely to occur.

The system can also use ancillary information to include the absolute speed of the aortic pulse wave. Individuals with low arterial compliance will have a decreased ability to manage intravascular changes. For example, a rigid pipe has no ability to compensate for volume changes. These individuals will have a higher pulse wave velocity so effective incorporation of this ancillary information will improve diagnostic performance of the system.

The process of creating intravascular volume changes can be accomplished in multiple ways. For example, changes in intravascular volume can be measured during dialysis. Passive leg raising is another method that moves blood volume from the legs into the central vasculature. Mechanical respiration, Valsalva, general breathing, placing the patient in the Trendelenberg position, lower body negative pressure chambers and administration of intravascular fluids all represent reasonable approaches to creating a defined change in intravascular volume.

The present invention has been described in the context of various example embodiments as set forth herein. It will be understood that the above description is merely illustrative of the applications of the principles of the present invention, the scope of which is to be determined by the claims viewed in light of the specification. Other variants and modifications of the invention will be apparent to those of skill in the art.

1. A method for assessing diabetes in an individual by characterization of a pulse waveform indicative of the response of a portion of the cardiovascular system of the individual to pressure pulses produced during the cardiac cycle, where “assessing diabetes” comprises determining the presence or likelihood of diabetes; the degree of progression of diabetes; a change in the presence, likelihood, or progression of diabetes; a probability of having, not having, developing, or not developing diabetes; the presence, absence, progression, or likelihood of complications from diabetes; or a combination thereof, the method comprising: determining a measure of arterial compliance of the individual from the pulse waveform; and assessing diabetes based on the measure of arterial compliance.

2. A method as in claim 1, wherein determining a measure of arterial compliance comprises obtaining a pulse waveform by noninvasive methods, and determining the measure of arterial compliance from the pulse waveform.

3. A method as in claim 2, wherein the pulse waveform is a photoplethysmogram.

4. A method as in claim 3, wherein determining the measure of arterial compliance comprises determining a measure of aortic compliance from the photoplethysmogram.

5. A method as in claim 3, wherein assessing diabetes comprises assessing diabetes based on the pulse waveform in combination with one or more of the following: age, gender, blood pressure, height, and measurements indicative of aortic length.

6. A method as in claim 3, wherein the photoplethysmogram is a photoplethysmogram acquired from a digit of the individual.

7. A system for assessing diabetes, where “assessing diabetes” comprises determining the presence or likelihood of diabetes; the degree of progression of diabetes; a change in the presence, likelihood, or progression of diabetes; a probability of having, not having, developing, or not developing diabetes; the presence, absence, progression, or likelihood of complications from diabetes; or a combination thereof, the system comprising:
(a) a light source configured to provide light suitable for illuminating tissue of the individual and having at least one wavelength affected by variance in the cardiac cycle of the individual;
(b) a detection system configured to detect light from the source, after it has interacted with tissue of the individual, for time spanning at least one cardiac cycle;
(c) a recording system configured to record information from the detection system;
(d) an analysis system configured to analyze the recorded information and assessing diabetes based on the characteristic of a primary wave and reflected waves in the pulse waveform.

8. A system as in claim 7, wherein the detection system is configured to associate detected light with at least two different average path lengths through the tissue.

8. A system as in claim 7, wherein the detection system is configured to detect light based upon polarization characteristics.

9. A system as in claim 8, wherein the detection system is configured to associate detected light with at least two different average path lengths through the tissue based on numerical aperture of optical elements in the detection system

10. An apparatus for diabetes assessment in an individual, where “diabetes assessment” comprises determining the presence or likelihood of diabetes; the degree of progression of diabetes; a change in the presence, likelihood, or progression of diabetes; a probability of having, not having, developing, or not developing diabetes; the presence, absence, progression, or likelihood of complications from diabetes, comprising: (a) an optical subsystem configured to determine a pulse photoplethysmogram of the individual; and (b) an input system, configured to accept biologic information comprising one or more of: gender of the individual, weight of the individual, waist circumference of the individual, history of disease of the individual's family, ethnicity, skin melanin content, smoking history of the individual, or a combination thereof; (c) an analysis subsystem, comprising a model relating pulse photoplethysmogram to a metric of diabetes assessment.

11. An apparatus as in claim 10, wherein the model comprises a classification relationship between diabetes assessment and pulse photoplethysmogram.

12. An apparatus as in claim 10, wherein the optical subsystem comprises a polarization system.

13. A method of determining a disease state in an individual, where “determining a disease state” comprises determining the presence or likelihood of diabetes; the degree of progression of diabetes; a change in the presence, likelihood, or progression of diabetes; a probability of having, not having, developing, or not developing diabetes; the presence, absence, progression, or likelihood of complications from diabetes, and where “determining a disease state” does not comprise measurement of glucose concentration, the method comprising:
(a) illuminating a portion of the tissue of the individual with light for a time spanning at least one cardiac cycle of the individual;
(b) detecting light emitted from the tissue for a time spanning at least one cardiac cycle of the individual;
(c) acquiring biologic information relating to the individual; and
(d) determining the disease state from the biologic information, the detected light, and a model relating biologic information, detected light, and disease state.

14. A method as in claim 13, wherein illuminating a portion of the tissue comprises illuminating a portion of the tissue with light having at least two wavelengths.

15. A method as in claim 13, wherein detecting light comprises detecting light that is responsive to pulse changes in the tissue.

16. A method as in claim 13, wherein the model determined parameters associated with arterial stiffness.

17. A method as in claim 13, wherein illuminating and detecting together produce a pulse photoplethysmogram.

18. A method as in claim 17 wherein the detected light is separated based upon polarization characteristics.

19. A method as in claim 18 wherein the detected light with different polarizations is used to determine degree of polarization.

20. A method as in claim 19 wherein the model for determination of arterial compliance uses the determined degree of polarization.
